(19) United States
(12) Patent Application Publication
(10) Pub. No.: US 2025/0288320 A1
(43) Pub. Date: Sep. 18, 2025

(54) A BIOMEDICAL DEVICE, A SYSTEM COMPRISING A BIOMEDICAL DEVICE, A METHOD FOR PERCUTANEOUS CATHETERIZATION OF A BODY DUCT, AND A METHOD FOR ALIGNING AN EXTERNAL IMAGING MEANS

(71) Applicant: Caranx Medical SAS, Paris (FR)

(72) Inventors: Jonas Victor Harmen SMITS, Bierbeek (BE); Eric SEJOR, Nice (FR); Giulio CERRUTI, Saint-Laurent-Du-Var (FR); Pierre BERTHET-RAYNE, Cagnes-sur-Mer (FR); Camille BENOIST, Saint Paul de Vence (FR)

(73) Assignee: Caranx Medical SAS, Paris (FR)

(21) Appl. No.: 18/863,117

(22) PCT Filed: May 1, 2023
(86) PCT No.: PCT/EP2023/061428

§ 371 Date: Nov. 5, 2024

(30) Foreign Application Priority Data
May 6, 2022 (EP) 22315100.2

(51) Int. Cl. A61B 17/34; A61B 17/00; A61B 17/3209; A61B 34/30; A61B 90/00; A61M 25/06; A61M 29/00; G16H 40/63

(52) U.S. Cl. CPC A61B 17/3415; A61B 17/32093; A61B 34/30; A61B 90/06; A61B 90/37; A61M 25/065; A61M 25/0662; A61M 29/00; G16H 40/63; A61B 2017/00017; A61B 2017/00398; A61B 2017/00477; A61B 2034/301; A61B 2090/064; A61B 2090/373; A61B 2090/376; A61B 2090/378; A61M 2025/0687

(57) ABSTRACT

A biomedical device for percutaneous catheterization of a body duct. The device comprising an introducer assembly which has a dilator for widening an insertion site of a patient to access the duct, a sheath for introduction into the duct, and an inner guiding unit for guiding the dilator and/or sheath into the duct. The dilator having a tapered outer section and a distal opening and being arranged at least partially concentrically around the guiding unit. The sheath having distal opening and being arranged at least partially concentrically around the dilator, such that the guiding unit, dilator, and sheath are movable relative to each other in a longitudinal direction of the introducer assembly. The dilator may be guided along the guiding unit without removal of a puncturing element for puncturing the duct at a proximal end of the introducer assembly after insertion of the puncturing element into the duct.

This application is a National Stage completion of PCT/EP2023/061428 filed May 1, 2023, which claims priority to European patent application serial no. 22315100.2 filed May 6, 2022.

### FIELD OF THE INVENTION

The invention relates to a biomedical device, a system comprising a biomedical device, a method for percutaneous catheterization a body duct, and a method for aligning external imaging means relative to a body duct.

### BACKGROUND OF THE INVENTION

Introduction of a catheter in a percutaneous procedure for the purpose of catheterization requires puncturing vascular body ducts, in particular arterial body ducts. This placement of a catheter in an arterial and/or venous system is often performed in the prior art by using the Seldinger technique.

The Seldinger technique comprises placing a cannula within the body duct and introducing a guidewire being circumferentially surrounded by the cannula through the cannula into the body duct. Subsequently, the cannula is retracted from a proximal end of the guidewire, such that a dilator and finally a catheter sheath can be advanced over the guidewire into the body duct.

U.S. Pat. No. 5,7493,71 A discloses a device for automated guidewire placement using the Seldinger technique with a hollow cannula with a cannula tip with gripping means for releasably engaging a guidewire.

N. Zevallos et al., “Toward Robotically Automated Femoral Vascular Access,” 2021 International Symposium on Medical Robotics (ISMR) discloses a robot-assisted arterial catheterization through the Seldinger technique.

A particularly time-consuming and complex step, in particular of a non-manual automated implementation of the Seldinger technique, is the removal of the cannula by fully retracting the coaxial cannula over the guidewire.

In general, the prior art lacks a simple, reliable, and time-saving method to enable percutaneous catheterization, in particular automated percutaneous catheterization, of a body duct.

### SUMMARY OF THE INVENTION

It is the object of the present invention to overcome these and other disadvantages in the prior art. In particular, the invention shall provide a device and system that reduces and/or optimizes the number of steps required for surgical catheterization, particularly catheterization using the Seldinger technique, and provides a standardized technique for atraumatic catheterization.

Another object of the invention is to provide a biomedical device and system with reduced complexity.

The object is solved by a biomedical device, a system comprising a biomedical device, a method for percutaneous catheterization, and a method for aligning an external imaging means according to the independent claims. Further embodiments are defined in the dependent claims.

A biomedical device according to the invention is used for percutaneous catheterization of a body duct, in particular arterial catheterization. The device comprises an introducer assembly which has a dilator for widening of an insertion site of a patient to access the body duct. The device further comprises a sheath for introduction into the body duct and an inner guiding unit for guiding the dilator and/or the sheath into the body duct. The dilator has a tapered outer section and at least one distal opening and is or can be arranged at least partially concentrically around the guiding unit. The sheath has at least one distal opening and is arranged at least partially concentrically around the dilator.

Once the puncturing element is inserted into the body duct, the dilator may be guided along the guiding unit without complete withdrawal of the puncturing element from a proximal end of the introducer assembly.

The proximal end refers to the end of the introducer assembly that is locatable outside of the patient proximally relative to an insertable distal end of the introducer as perceived by a surgeon.

The introducer assembly can be a consumable introducer assembly having pre-aligned movable parts for usage with a robotic actuation.

The guiding unit can comprise or consist of a guidewire, the puncturing element, and/or an insertion guide.

The puncturing element can be a cannula, optionally a commercially available cannula with a hollow cylindrical cannula shape and a sharpened distal end which is sharpened with an oblique grind.

The tapered outer sections of the dilator and sheath can be configured for atraumatic introduction into the body duct over the guiding unit.

The guiding unit can comprise an insertion guide for inserting the puncturing element or the guidewire. The insertion guide has a distal section and a proximal section. The proximal section has a proximal lumen for at least partially receiving the puncturing element or the guidewire. The distal section has a distal lumen for receiving the puncturing element or the guidewire. The distal lumen and the proximal lumen are connected to each other.

In this embodiment, the distal section has a lateral opening at a proximal end through which the puncturing element can be reversibly inserted into the distal lumen and a distal opening from which the puncturing element can be inserted into the body duct.

The distal section of the insertion guide can be insertable into the body duct with the puncturing element projecting from its distal opening.

The puncturing element does not need to be fully retracted from the proximal end of the introducer assembly but instead can be retracted from the lateral opening which is arranged at an intermediate position. Thereby, the catheterization is made easier. In addition, the insertion guide can be inserted into the body duct simultaneously with the puncturing element which further simplifies the catheterization procedure.

The openings of the device, in particular the distal opening, the lateral opening, the at least one distal opening of the dilator and/or the sheath can comprise a hemostatic valve for reducing leakage and/or blood backflow.

The distal lumen of the insertion guide can extend in a straight line between the lateral opening and the distal opening of the distal section.

Alternatively, the distal section of the insertion guide can be elastically deformable, such that the distal lumen can assume the shape of a straight line between the lateral and distal opening.

The distal lumen of the insertion guide extending in a straight line allows for easy insertion and removal of a puncturing element which may have a straight shape and which may not be readily bendable.

The length of the distal lumen in a longitudinal direction of the insertion guide is preferably sufficient to reliably guide the puncturing element into the body duct and short enough to allow rapid insertion and removal of the puncturing element which is typically in the form of a cannula. The length of the distal lumen can range from 1 cm to 20 cm, preferably 7 cm to 20 cm.

A longitudinal axis of the proximal section of the insertion guide can be arranged or arrangeable at an angle of 1° to 125°, preferably an angle of 10° to 75°, particularly 20° to 50°, relative to a longitudinal axis of the distal section of the insertion guide.

Such an angle allows for reliable insertion of the puncturing element and at the same time allows a guidewire to be insertable through the proximal and distal lumen of the insertion guide. The guidewire is sufficiently flexible to conform to the angle between the distal and proximal section of the insertion guide.

The dilator can be arranged or arrangeable coaxially around the insertion guide and configured to be advanced relative to the insertion guide in the longitudinal direction of the insertion guide.

The dilator can also be arranged or arrangeable coaxially around a guidewire and configured to be advanced relative to the guidewire in a longitudinal direction of the guidewire. A mantle section of the proximal and distal section can then have a release opening extending in a longitudinal direction. The release opening can be permanently open or openable. The release opening is configured for radially releasing the guidewire which is at least partially arranged or arrangeable in the proximal and distal lumen.

A dilator arranged coaxially around the insertion guide and advanceable relative to the insertion guide in the longitudinal direction allows for easy introduction of the dilator into the body duct over the proximal and distal section of the insertion guide.

The distal and proximal sections of the insertion guide can have a constant cross-sectional dimension along their length, so that the dilator can be advanceable over the complete insertion guide.

The insertion guide can extend at least partially, in particular completely, through an inner lumen of the dilator.

A dilator arranged coaxially around the guidewire and advanceable relative to the guidewire in the longitudinal direction allows for using existing commercially available components for the dilator, the sheath, and the guidewire which are used for performing the Seldinger technique. The guidewire can be removed radially from the introducer guide, such that the introducer guide does not have to be removed through the proximal end of the device. Thus, the guidewire can stay in place and the introducer guide can be removed from the patient's site before the dilator is advanced over the guidewire to insert the introducer assembly.

The dilator and/or the sheath can comprise a hydrophobic coating which allows for an atraumatic insertion into the body duct.

The dilator and/or the sheath can have a size between 1 Fr and 34 Fr, in particular 3 Fr to 16 Fr, wherein 1 Fr is equal to 0.33 mm.

The dilator and/or the sheath can comprise or consist of thermoplastics such as silicones, polyethylene, polypropylene, polyetheretherketone, fluoropolymers, polyvinyl chloride and/or polyurethane.

The device can comprise multiple dilators and/or sheaths.

A guidewire of the guiding unit can be pre-aligned within a proximal lumen of a proximal section of an insertion guide of the guiding unit.

The guiding unit and in particular the insertion guide, the guidewire, and/or the puncturing element, can comprise or consist of a metal, in preferably stainless steel or a shape memory alloy, particularly nitinol.

The puncturing element can have a hollow cylindrical cannula-shape.

The puncturing element can comprise at least one retaining structure. The retaining structure can have a recess on an outer surface and open radially outwardly for receiving a guidewire extending at least partially along a longitudinal direction of the puncturing element.

The retaining structure may also have a lumen defined by a wall which is at least partially open or openable for radially releasing a guidewire which is at least partially arranged or arrangeable in the lumen.

The retaining structure can alternatively have an attachment mechanism for attaching a guidewire in parallel to the puncturing element.

The retaining structure can comprise a retaining state for retaining the guidewire at the puncturing element and a releasing state for radially releasing the guidewire from the lumen or recess of the puncturing element.

The retaining structure can be convertible, optionally reversibly convertible, from the retaining state to the releasing state, e.g. by a rotational movement or a translational movement of the retaining structure and/or the guidewire relative to each other or to the introducer assembly.

The retaining structure can comprise a spring-loaded tip of the puncturing element. The spring-loaded tip can be converted from the retaining state to the releasing state. The spring-loaded tip is convertible to the retaining state when compressed and to the releasing state when decompressed. Thereby, the spring-loaded tip can retain the guidewire during introduction through skin and tissue into the body duct while being compressed and can release the guidewire within the body duct based on a reduction of the compressive force within the body duct.

The retaining structure can comprise a mechanical coupling element to frictionally engage the puncturing element, an, optionally bioresorbable, glue, or a lumen within the puncturing element for applying a local suction to retain the guidewire. The guidewire may have a lumen for applying the local suction for retaining the guidewire.

The retaining structure can be convertible by applying local vibration to overcome frictional engagement of the mechanical coupling element. The retaining structure can also be convertible by eliminating a connection provided by the, preferably bioresorbable, glue. The retaining structure may be convertible from a retaining state, in which suction is applied to the lumen of the puncturing element or guidewire, to a releasing state by ceasing local suction on the lumen.

Thereby, the retaining structure may be converted to the releasing state for releasing the guidewire once the guidewire has been inserted into the body duct.

The open or openable lumen can comprise at least one, in particular two, mantle sections. A first and a second mantle section can be movable relative to each other. The first and second mantle sections can form a C-shape, in particular form a C-shape with different diameters, such that one of the mantle sections can be positioned to be at least partially enclosed by the other of the mantle sections.

The recess or lumen can be arranged partially along the longitudinal direction and partially in a circumferential direction of the puncturing element, in particular along a helical or curved path. Such a helical or curved path can impede movement of the guidewire in the longitudinal direction of the puncturing element relative to the puncturing element due to increased friction.

To increase a static and/or a sliding friction, the recess or the lumen of the puncturing element can be configured to frictionally engage the guidewire.

The puncturing element can be relatively long and can have a length of 6 cm to 35 cm, preferably 8 cm to 30 cm, more preferably 12 cm to 25 cm. The proximal end of the puncturing element can have a connection to a distal end of the guidewire, such that the puncturing element and the guidewire are essentially flush with each other.

A relatively long puncturing element allows to introduce the dilator into the body duct coaxially around the puncturing element directly, in particular without requiring a guidewire.

A puncturing element connected to the distal end of the guidewire has the advantage, that the longitudinal length of the puncturing element can be shorter than 6 cm, less than 2 cm, preferably less than 1 cm. Due to a shorter puncturing element which is rather stiff, the dilator can be advanced more reliably along the more flexible guidewire section when it is introduced into the body duct.

The puncturing element and guidewire can each have different elastic properties. The guidewire can e.g. have a lower bending stiffness than the puncturing element such that a curvature of the guidewire can be adapted to the body duct and/or the introducer assembly more easily.

The guidewire connected to the puncturing element can still function as a push rod which can be moved in the longitudinal direction relative to the introducer assembly to introduce the puncturing element. The dilator can be introducible into the body duct coaxially around the puncturing element which is connected to the guidewire.

A puncturing element connected to the distal tip of the end of the guidewire allows to avoid the traditional steps of the Seldinger technique, i.e. separately introducing the guidewire after introduction of the puncturing element and retracting the puncturing element once the guidewire was introduced into the body duct.

The puncturing element can be connected or connectable to the dilator and comprise an actuating element, in particular a spring, which is convertible between a retracted state and an extended state. By conversion of the actuating element, a puncturing section of the puncturing element for contacting and puncturing tissue is retractable behind a non-puncturing surface, or a non-puncturing surface can be advanced for shielding the puncturing section.

The puncturing element can then be used for piercing tissue and otherwise be rendered atraumatic by the non-puncturing surface. In addition, the dilator can be directly introduced by using the puncturing element which has a non-puncturing surface shielding the puncturing surface, without necessarily requiring a guidewire and/or an insertion guide.

The non-puncturing surface can be a located on a distal end of the dilator or on a separate element of the introducer assembly.

The non-puncturing surface and/or the actuating element may be arranged concentrically within the lumen of the puncturing element. The puncturing element may in particular be a Veress cannula.

The puncturing element can comprise an anchoring member arranged at least partially circumferentially on the puncturing element and preferably forming a longitudinal section of the puncturing element. The anchoring member comprises a collapsed configuration and an expanded configuration. The collapsed configuration has a radial dimension which is smaller than a radial dimension in the expanded configuration.

In the expanded configuration the anchoring member can prevent advancement of the puncturing element into the patient, in particular into the body duct, beyond a predetermined longitudinal distance of the puncturing element.

The anchoring member in the expanded configuration can additionally or alternatively prevent retraction of the puncturing element from the body duct beyond a predetermined longitudinal distance of the puncturing element, in particular by contacting an inner wall of the body duct. This can provide secure anchoring of the puncturing element within the body duct and prevent accidental removal of the puncturing element from the body duct.

Such an anchoring member prevents potential injuries caused by inserting the puncturing element too deeply into the skin of a patient or the body duct of the patient. In addition, such a puncturing element can be used for direct insertion of the dilator over the puncturing element, so that a guidewire and/or an insertion guide are not necessarily required.

The predetermined longitudinal distance may be shorter than the width of the body duct or greater than the distance from a puncture position of the puncturing element on a patient's skin to the body duct. The anchoring member can contact the skin of the patient or an outer wall of the body duct in the expanded configuration.

The dilator can comprise an anchor actuator, such that upon longitudinal movement of the dilator relative to the puncturing element, the anchor actuator engages the anchoring member in the expanded configuration to convert the anchoring member from the expanded configuration to the collapsed configuration.

The dilator can comprise longitudinal slots arranged neighboring the distal opening of the dilator. The longitudinal slots are configured to be advanced past the anchoring member in the longitudinal direction of the puncturing element.

The anchor actuator may comprise a radially extending protrusion arranged at least partially circumferentially on the puncturing element and spaced by a predetermined longitudinal distance from the anchoring member, such that the protrusion can be engaged by the dilator with slots being advanced over the anchoring member.

The anchoring member can be biased toward the expanded configuration, in particular biased due to a restoring element, in particular by a spring element, which exerts a restoring force on the anchoring member. Thereby, the anchoring member assumes the expanded configuration as a default state when not being retained or engaged by the anchor actuator of the dilator.

The sheath, the dilator, and/or at least a part of the guiding unit, in particular the puncturing element, can be connected to at least one mechanical coupling. A relative longitudinal movement of one of these components (the sheath, the dilator, and/or at least the part of the guiding unit) beyond a predetermined point causes the coupling to move another of these components in the longitudinal direction.

The coupling allows the movement of the components of the introducer assembly, in particular the dilator, the sheath, and at least the part of the guiding unit, in particular the puncturing element, to be coupled or decoupled with each other.

The coupling may be configured to couple the components only mono-directionally, in particular in the distal direction or proximal direction of the introducer assembly, or couple the components bidirectionally, in the proximal direction and the distal direction of the introducer assembly.

The coupling can comprise at least one, in particular two, coupling elements.

The coupling can comprise a first coupling element located on one of the sheath, the dilator, or the part of the guiding unit and a second coupling element located on the other of the sheath, the dilator, or the part of the guiding unit. The coupling elements can be formed to positively engage each other at the predetermined point.

The coupling can comprise at least one recess and/or at least one protrusion extending in the radial direction.

The recess and/or the protrusion can be arranged at least partially in a circumferential direction of the dilator, sheath or at least the part of the guiding unit, in particular the puncturing element.

In one embodiment of the invention which only requires a single actuation unit configured for actuation of the guiding unit, a plurality of couplings can be arranged to allow insertion of the sheath via the dilator and the guiding unit and retraction of the dilator and the guiding unit. This can be achieved by the dilator comprising two coupling elements for engaging a coupling element of the guiding unit, in particular puncturing element, and preferably one coupling element for engaging a coupling element of the sheath.

The coupling elements of the dilator and guiding unit can be arranged such that advancement of the guiding unit in the distal direction beyond a first pre-determined position results in advancement of the guiding unit and dilator in unison. In addition, the coupling elements can be arranged, such that retraction of the guiding unit in the proximal direction beyond a second pre-determined position results in a retraction of the guiding unit and dilator in unison.

The dilator may have a coupling element which allows to couple a distal movement of dilator to the sheath by engaging a coupling element of the sheath while retraction of the dilator is independent from the movement of the sheath.

The invention further relates to a system for carrying out a percutaneous catheterization of a body duct, in particular an arterial catheterization, through an insertion site of a patient's skin using a biomedical device having an introducer assembly, in particular a biomedical device previously described. The introducer assembly has a dilator for widening of an insertion site of a patient to access the body duct, a sheath for introduction into the body duct, and an inner guiding unit for guiding at least one of the dilator and the sheath into the body duct. The dilator has a tapered outer section and at least one distal opening and is arranged at least partially concentrically around the guiding unit. The sheath has at least one distal opening and is arranged or arrangeable at least partially concentrically around the dilator. The guiding unit, the dilator, and the sheath are movable relative to each other in a longitudinal direction of the introducer assembly. The system comprises at least one actuation unit which is configured to actuate at least one of, in particular two of, preferably all of the dilator, the sheath, and the guiding unit, in particular at least one of a puncturing element, and insertion guide, and/or a guidewire of the guiding unit of the device in the longitudinal direction. The system comprises a shearing unit which is actuatable to perform a sheared aperture at an insertion site, in particular an incision extending from or across the insertion site. As used in the present application, shearing refers to any separation of parts of tissue by tools such as a scalpel, scissors, or any other instruments for sharp or blunt dissection.

The guiding unit can comprise or consist of a guidewire, an insertion guide, and/or the puncturing element.

The puncturing element can be a cannula, optionally a commercially available cannula with a hollow cylindrical cannula shape and a sharpened distal end which is sharpened with an oblique grind.

The shearing unit allows for widening the insertion site, in particular an incision of a cutting element of the shearing unit, to allow an atraumatic widening/insertion of the dilator and/or sheath into the body duct of a patient.

The shearing unit may be arranged at a distal end of the system, such that the shearing unit can be arranged adjacent the distal end of the biomedical device, in particular adjacent to a distal end of the puncturing element.

The shearing unit may be mechanically or functionally coupled with at least a part of the introducer assembly, in particular the dilator or sheath, and preferably the inner guiding unit formed by a puncturing element. This coupling allows that the advancement of the part of the introducer assembly causes actuation of the shearing unit in a fully automated manner, in particular to perform the incision. The coupling is preferably configured such that the incision via a cutting element of the shearing unit is performed sequentially after insertion of the inner guiding unit, in particular the puncturing element, at the insertion site. This facilitates carrying out the percutaneous catheterization using the system.

The cutting element of the shearing unit can be configured to be actuatable in such a way that the incision originates from a puncturing hole formed by the puncturing element at the insertion site. This allows to perform the incision with better precision while the inner guiding unit, e.g. the puncturing element, is inserted at the insertion site.

The system can have at least one mounting section. The mounting section is adapted for releasably mounting at least one part of the biomedical device, in particular the dilator, the sheath, and the guiding unit, preferably the puncturing element, the guidewire, and/or an insertion guide. The mounting section may comprise an at least partially open shape in a radial direction of the system corresponding to the mountable part of the biomedical device.

The system may have a plurality of mounting sections for mounting more than one, in particular all, of the sheath, the dilator, and the inner guiding unit separately. This allows improved mounting, coupling and individual as well as collective actuation of these elements by the actuation unit.

The plurality of mounting sections can be configured to receive the sheath, the dilator and/or the inner guiding unit with a specific predetermined position/orientation with respect to each other. This allows the introducer assembly to be positioned in an optimized pre-operational orientation to facilitate the catheterization and enables improved actuation considerations.

The plurality of mounting sections may comprise a coupling, optionally a plurality of coupling elements, such that the movement of the mounting section may be coupled with respect to each other. Analogous to the coupling of the biomedical device, this can allow for simplifying the actuation of the introducer assembly via an actuation unit, e.g. when advancing or retracting one or a plurality of the sheath, the dilator, and the guiding unit.

The system can have an engagement member which is convertible between an engagement configuration and a disengagement configuration. The at least one mountable part of the biomedical device in the engagement configuration is fixedly held within the mounting section, at least with respect to movement along the longitudinal direction, in particular with respect to a radial direction of the biomedical device, relative to the mounting section. In the disengagement configuration the at least one mountable part is detached from the mounting section, such that the mountable part can be removed from the system.

Such an engagement member enables secure attachment of the biomedical device in an anteriorly defined position so that the percutaneous procedure can be performed reliably and reproducibly.

The actuation unit may be adapted to actuate the at least one mountable part of the biomedical device, e.g. the dilator, the sheath, and/or the guiding unit, by moving the mounting section with the open shape for receiving the mountable part of the introducer assembly.

The engagement member may be based on a form-fitting mechanism, e.g. a snap-fit mechanism, a locking mechanism, e.g. a closable bracket or a clamp.

The engagement member may be integrally formed with the mounting section, in particular the open shape of the mounting section. This may e.g. allow for manual conversion of the engagement member between the engagement configuration and disengagement configuration.

Alternatively, the engagement member may be actively convertible, e.g. via a processing unit controlling an actuator. This allows for a fully automated process that does not depend on the skills of a clinician, while minimizing a risk of injury when detaching the system from the mountable part of the introducer assembly.

The system may comprise exactly one actuation unit. A single actuation unit simplifies the design and construction of the system and greatly reduces the associated costs.

The actuation unit may be configured to actuate one of the dilator, the sheath, and the guiding unit while remaining stationary with respect to the system. Alternatively, the actuation unit may be configured to actuate one of the dilator, the sheath, and the guiding unit by translationally moving itself, in particular along a longitudinal direction of the system.

The actuation unit can comprise a clutch, in particular a clutch comprising at least two rollers which have an adjustable interdistance. Thereby, the actuation of the actuation unit can be switched between configuration for actuating at least two, preferably three, in particular preferably all of the dilator, the sheath, and the guiding unit, in particular the puncturing element, the guidewire, and/or the insertion guide.

The system can comprise at least one guiding element, in particular at least two guiding elements, preferably formed by a guiding rail, such that at least one of the actuation unit and the at least one mounting section are movable along the guiding element.

The actuation unit and/or the mounting section can be connected or connectable to at least one guiding element, in particular two guiding elements simultaneously. This enables accurate and reliable guidance along the guiding elements in the desired orientation.

The guiding elements can be arranged or arrangeable in the longitudinal direction of the system. The at least two, in particular three, preferably four, guiding elements can be arranged parallel with respect to each other.

The guiding elements can have one end which is connected or connectable to a base of a system and another free end without any connections.

The shearing unit may be configured to be actuatable by a specific movement of the actuation unit or mounting section, in particular a specific movement exceeding a specific spatial position, preferably at a distal end of the system.

The system can comprise a processing unit which is adapted for control of at least one of, preferably at least two of or all of, the shearing unit, the actuation unit, and the engagement member.

Control by a processing unit enables precise coordination of the system for performing the catheterization, in particular coordination based on external imaging means, external guidance means, or coordination between the actuation unit and the shearing unit.

According to another aspect of the invention a system for carrying out a percutaneous catheterization of a body duct, in particular a previously described system, comprises a shearing unit. The shearing unit comprises at least one, in particular two, shearing elements for performing a sheared aperture at an insertion site. The system comprises an actuation unit which is configured for translationally moving the at least one shearing element, in particular translationally moving the at least one shearing element parallel to a longitudinal axis of the shearing unit. The actuation unit is configured for moving the shearing unit to perform the sheared aperture by (i) rotating the at least one shearing element around a longitudinal axis of the shearing unit or (ii) moving the shearing elements radially outwardly with respect to a longitudinal axis of the shearing unit.

This allows performing a spatially precise sheared aperture or plurality of sheared apertures at the insertion site of the patient's tissue for carrying out the percutaneous catheterization in a simple and reliable manner.

The system may further comprise a processing unit which is adapted for operating the shearing unit via the actuation unit.

The system may comprise a processing unit which is configured for operating the actuation unit and the shearing unit by repeated sequential translational movement of the shearing unit in a distal direction of the shearing unit and subsequent rotation of the shearing unit around the longitudinal axis. The rotation of the shearing unit may not exceed an angle of 360°. Preferably a rotation of the shearing unit does not exceed an angle of 180° in a first angular direction and subsequent rotation of the shearing unit in a second angular direction, opposite to the first angular direction does not exceed an angle of 180°. The at least one shearing element has a non-rotationally symmetric cross-sectional shape at its distal tip or is arranged radially offset to a rotational axis of the shearing unit. The at least one shearing element is further adapted for performing a sheared aperture by blunt dissection.

Alternatively, the shearing element may be convertible to have a non-rotationally symmetric cross-sectional shape at the distal tip.

This sequential translational movement and rotational movement of the shearing unit allows to perform a blunt dissection for performing a catheterization with enhanced precision and control based on the central rotational trajectory while minimizing tissue trauma.

The system may comprise a blunt inserter which has a blunt distal tip, in particular a rounded distal tip, and is configured to be insertable into an inner lumen of the shearing unit.

This blunt inserter enhances the stiffness of the shearing unit by providing increased structural integrity when performing the sheared apertures.

The processing unit and actuation unit may be adapted for advancing and retracting the blunt inserter through the inner lumen of the shearing unit in a synchronized manner for performing the sheared apertures.

The system may comprise an expansion element which is arrangeable between the first and second shearing elements. The expansion element may be formed by an inflatable expansion element or an expansion element which is configured to be insertable into an inner lumen of the shearing unit. The expansion element is configured for moving the shearing elements radially outwardly with respect to the longitudinal axis of the shearing unit.

This expansion element allows for selectively radial expansion of the shearing elements to perform a sheared aperture at the insertion site.

The blunt inserter may form the expansion element and be configured for moving the shearing elements, in particular only distal ends of the shearing elements, radially outwardly by being translationally moved through the inner lumen in the proximal or the distal direction, e.g. similarly to a dowel. The blunt inserter may comprise a longitudinally varying cross-sectional dimension, in particular at its distal tip, to facilitate the radial movement of the shearing elements.

The shearing elements may be pivotally connected at at least one fulcrum point for relative pivotal movement thereabout. A conversion mechanism of the shearing unit may be configured for converting a translational movement, in particular a translational movement parallel to a longitudinal axis of the shearing unit, of an actuation section of the conversion mechanism into an outwardly pivoting movement of the shearing elements around the at least one fulcrum point for performing the sheared aperture.

The outward pivoting movement of the shearing elements enhances compatibility of the system, since the sheared apertures(s) can be used for insertion or even sequential insertion of medical instruments in a simple and effective manner.

The shearing unit may comprise a plurality of fulcrum points, in particular adapted for coupling the pivoting movement of the first and second shearing elements. In a preferred embodiment, moving the first shearing element results in an equidistant movement of the second shearing element in the opposing direction based on the coupling of pivoting movements.

The actuation unit may be configured to actuate the conversion mechanism to convert the shearing unit between a closed configuration in which the shearing elements are adjacent each other and an open configuration in which the shearing elements are pivoted outwardly. The actuation unit can be adapted to advance and retract the shearing unit towards and away from the insertion site.

This allows a simple machine-controlled conversion of the shearing unit, in particular in a fully automated manner.

The conversion mechanism may be shaped to be longitudinally expandable and contractible. The conversion mechanism may be adapted, such that in the open configuration of the shearing elements, the conversion mechanism is in a longitudinally contracted position and in the closed configuration of the shearing elements the conversion mechanism is in a longitudinally expanded position.

The processing unit and the actuation unit may be adapted for operating the actuation unit and the shearing unit by repeated sequential advancement of the shearing unit in the longitudinal direction of the shearing unit in the closed configuration and subsequent conversion to the open configuration for performing a plurality of sheared apertures at an insertion site.

This allows for sequentially performing sheared apertures, preferably blunt dissection sheared apertures, through the tissue in a distal direction of the shearing unit.

The system may further be configured for advancement, in particular sequential advancement, of medical instruments, such as the previously described biomedical device, through the sheared apertures to a body duct.

The system may comprise a previously described biomedical device, optionally having a dilator, a sheath, and/or an inner guiding unit which are movable by the system, in particular by a previously described actuation unit of the system in a longitudinal direction of the biomedical device.

The actuation unit which is adapted for advancing/retracting the shearing unit may be adapted to advance/retract the shearing unit in a direction parallel to the longitudinal direction, in particular along the longitudinal direction of the biomedical device.

Alternatively, the actuation unit for advancing/retracting the shearing unit may be adapted for translationally moving the shearing unit along an angled direction with respect to the longitudinal direction of the biomedical device, e.g. angled by more than 5°, 15°, 25°, or 35°, in particular along a guiding element, such as a guiding rail. The angled direction of movement may be aligned to intersect the longitudinal direction at a distal section of the system corresponding to a position of the insertion site.

By angling the direction of movement of the shearing unit in this manner, the shearing unit can be easily moved out of the way after the performing of a sheared aperture to advance the introducer assembly of the system for catheterization of the insertion site along the longitudinal direction.

The shearing unit may be adapted for performing the sheared aperture by carrying out the outward pivoting movement of the shearing elements in a common plane.

Alternatively, the shearing unit may be adapted for performing the sheared aperture by carrying out a blunt dissection via the shearing elements. The blunt dissection may minimize tissue trauma by reducing the risk of damaging nearby bodily structures, nerves, or vessels and consequently enhances wound healing due to less inflammation, complications, such as infections, and tissue scarring.

The first and second shearing elements may have distal ends which converge to form at least one distal tip, in particular a sharp distal tip, for insertion to the patient's tissue at an insertion site when the first and second shearing elements are in a closed configuration.

This allows precise positioning of the shearing unit and system on the patient's tissue at the insertion site in the closed configuration when performing the outwardly pivoting movement to an open configuration to perform the sheared aperture(s).

Alternatively, the first and/or second shearing elements may have a T-shaped distal tip respectively for performing a blunt dissection by spreading the tissue at the insertion site. The T-shaped distal tips of the shearing elements can be placed in a lateral direction parallel to each other, in particular adjacent to each other, on the tissue of a patient to perform the blunt dissection.

The shearing unit may be adapted such that a longitudinal axis of the distal tip is essentially arranged along a longitudinal axis of an introducer assembly of the system.

The shearing elements may be configured such that respective blunt edges or cutting edges of the shearing elements face radially outwardly relative to each other in a closed configuration.

Blunt outwardly edged shearing elements of the shearing unit enables a less invasive approach to blunt dissection by reducing the tissue trauma.

The cutting edges of the shearing elements allow performing an incision which may be carried out outwardly with respect to the insertion site prior to performing the catheterization.

The first and second shearing elements having blunt or cutting edges may be arranged in a common plane of radially outward pivotable movement with respect to the longitudinal direction o f the shearing unit.

The system can comprise the biomedical device, in particular a biomedical device as previously described.

A system according to the invention comprises a biomedical device, preferably a previously described biomedical device, for percutaneous catheterization of a body duct, in particular arterial catheterization. The biomedical device comprises an introducer assembly which has a dilator for widening of an insertion site of the patient to access the body duct, a sheath for introduction into the body duct, and an inner guiding unit for guiding the dilator and/or the sheath.

The dilator has a tapered outer section, a distal opening, and is arranged at least partially concentrically around the guiding unit. The sheath has a distal opening and is arranged at least partially concentrically around the dilator.

The guiding unit, the dilator, and the sheath are movable relative to each other in a longitudinal direction of the introducer assembly. The system comprises at least one actuation unit which is configured to actuate the dilator, the sheath, and/or the guiding unit, in particular a puncturing element, a guidewire, and/or an insertion guide, of the device. The actuating movement is a movement in a longitudinal direction of the introducer assembly.

The system can comprise at least one actuation unit, preferably exactly one actuation unit, which comprises a clutch, in particular a clutch comprising at least two rollers which have an adjustable interdistance. The actuation of the actuation unit can be switched between configurations for actuating the dilator, the sheath, and/or the guiding unit, in particular the puncturing element, the guidewire, and/or an insertion guide.

The rollers can be configured for frictionally engaging the dilator, the sheath, and/or the guiding unit.

According to another aspect of the invention, a system comprises a biomedical device, preferably a previously described biomedical device, for percutaneous catheterization of a body duct, in particular arterial catheterization. The device comprises an introducer assembly for puncturing an application site at a patient's skin. The introducer assembly comprises a positionable and actuatable cannula for puncturing the skin at a desired place and with a desired direction.

The device comprises an external imaging means, in particular an ultrasound probe, e.g. a Doppler ultrasound probe for measuring a blood flow of the body duct, or a X-ray imaging probe. The imaging means are configured for imaging a body duct of a patient, in particular an arterial body duct, in two dimension or three dimensions, optionally in a time periodic measurement. The external imaging means and the introducer assembly are connected to a common support of the system.

The external imaging means and the introducer assembly, in particular the guiding unit, can be configured to be fixedly pre-oriented with respect to each other. A longitudinal axis of the field of view of the external imaging means and the introducer assembly may be arranged pre-oriented at an angle of 1° to 90°, in particular 20° to 60°, in particular preferably 30° to 45°, to each other.

The angle, a translational movement, and/or a rotational movement, between the imaging means and the introducer assembly may be adjustable by an alignment element, in particular at least one clamp or screw.

Additionally, the imaging device may be configured to image at least one other anatomical feature and generate data based on this other anatomical feature for the purpose of path planning. The anatomical feature may also be provided by or a preoperative scan for this purpose. The anatomical feature may be the position and orientation of nerves, ligaments, bones, fat, muscle, organ tissue, bifurcations, arterial walls, and calcifications.

The system can comprise an external guidance means. The external guidance means can comprise a stereo camera or a RGBD camera, a LIDAR system, and/or an electromagnetic sensor. Based on these guidance means a distance of the patient, in particular the body duct, and/or a surrounding of the patient to the imaging means and/or the introducer assembly, in particular the guiding unit, can be determined or reconstructed.

The system may have a landmark which comprises or consists of one or a plurality of visual recognition elements, in particular fiducials. The visual recognition elements can have a particular shape, in particular a line shape, a polygon shape or circular shape.

The plurality of visual recognition elements can have a predetermined distance to each other, such that the guidance means can be calibrated by measuring the distance of the visual recognition elements from the guidance means.

The visual recognition elements, in particular a patch, may be applied to or arranged on a drape or attached to the patient, in particular close to an insertion site of the patient to access the body duct.

The guidance means can be immovably connected to the external imaging means, immovably connected to the patient or a stationary surface, and/or attached to a movable surface which is independently movable from the imaging means.

The device of the system can comprise an introducer positioner for positioning the introducer assembly relative to the body duct and/or the common support. The introducer positioner can be configured to position the introducer assembly in two translational degrees of freedom and at least one rotational degree of freedom, or three translational degrees of freedom and at least one rotational degree of freedom, in particular two or three rotational degrees of freedom.

The common support of the system can be a hand-held support for manual alignment of the device, in particular the imaging means, relative to the patient.

It can also be a spatially fixed support which comprises a prepositioning means, in particular an adhesive, a strap, or a mechanically fixed or fixable carrier.

Alternatively, it can also be a spatially movable support which comprises a device positioner for positioning the device relative to the body duct independently in two or three translational degrees of freedom and at least one rotational degree of freedom, in particular one, two, or three rotational degrees of freedom.

The device positioner and/or the introducer positioner allows to align the introducer assembly, in particular the guiding unit, in translational and rotational degrees of freedom at the insertion site, in particular to a predetermined orientation relative to the body duct and/or the at least one visual recognition element.

The device positioner may be configured, preferably by the processing unit, to align the external imaging means with respect to the visual recognition element, in particular a visual recognition element placed on or in a proximity of the body duct.

The external imaging means being placed on or in the proximity of the body duct may be configured, preferably by the processing unit, to locally image the body duct, such that the processing unit can determine an insertion path based on the imaged body duct.

The introducer assembly may be positionable, preferably by the introducer positioner, based on the determined insertion path with respect to the body duct, such that the previously described insertion can be performed.

The predetermined orientation of the longitudinal direction of the introducer assembly, in particular the guiding unit, relative to the body duct may be at an angle of 1° to 80°, in particular 5° to 60°, preferably 10° to 55° to an axial direction of the body duct.

The introducer positioner and/or the device positioner can comprise at least one driving element, in particular an electric motor, a hydraulic actuator, a pneumatic actuator, and/or a piezo motor.

The system can comprise a force sensor which is configured for converting an external force, in particular an external torque, exerted on at least a subsection of the system, in particular a distal tip of the guiding unit, preferably a distal end of the puncturing element of the guiding unit, to an electrical signal indicative of the external force.

Such a force sensor can verify the position and/or orientation of the subsection of the system, in particular the puncturing element, relative to a patient or the body duct.

The system can comprise a processing unit for processing data of the measured image of the body duct, in particular a plurality of images, of the external imaging means.

Alternatively or additionally, the processing unit can process the determined or reconstructed distance, in particular a plurality of distances, of the guidance means.

It can also process the electrical signal, in particular a plurality of electrical signals, of the force sensor. The data can be processed by the processing unit for calculating at least one electrical signal for operating the introducer positioner and/or the device positioner.

Information derived from the electrical signal processed by the processing unit can alternatively or additionally be displayed on a display unit of the system.

The data of the imaging device relative to the at least one other anatomical feature may be processed by the processing unit for determining at least one signal for operating the introducer positioner and/or the device positioner.

The processing unit can be configured for operating the introducer positioner in a partially or fully automated manner, in particular requiring only partial or no manual operating input of a user.

The system can comprise a memory unit connected or connectable to the processing unit for saving data of the measured image of the body duct, in particular a plurality of images, of the external imaging means. Additionally or alternatively, the memory unit can be configured for saving data of the determined or reconstructed distance, in particular a plurality of distances, of the guidance means.

It can also be configured for saving data of the electrical signal, in particular a plurality of electrical signals, of the force sensor.

The data can be saved at an initial point in time. The processing unit can further be configured for retrieving the saved data at a later point in time from the memory unit for operating or supporting the operation of the introducer positioner and/or the device positioner.

The memory unit can be configured for saving the data of a preoperative scan or reconstruction, in particular a CT, MRI, or ultrasound scan or reconstruction.

The processing unit may be configured to register the preoperative or an intraoperative scan or reconstruction, in particular a CT, MRI, or ultrasound scan or reconstruction, optionally stored on the memory unit, to the image generated by the external imaging means.

The processing unit can be configured for using the saved and/or processed data to reconstruct an occupancy map which detects free spaces without obstacles.

The processing unit may be configured for determining the position of the insertion site and/or orientation of the introducer assembly, in particular the guiding unit, to avoid or minimize interference with calcifications within the body duct. The calcifications may be detected by the imaging means or the preoperative scan, particularly a CT scan.

The processing unit can be configured for processing the saved and/or processed data using an artificial intelligence, in particular by unsupervised, supervised, or reinforcement machine learning.

The guiding unit of any of the previously described systems can comprise a puncturing element which has a length of 6 cm to 35 cm, in particular 8 cm to 30 cm, preferably 12 cm to 25 cm. This allows the puncturing element which is at least partially surrounded by the dilator to guide the dilator into the body duct for catheterization without prior advancement of a guidewire and/or retraction of the puncturing element. The puncturing element and the dilator may be introduced into the body duct in unison with a predefined spatial relationship to each other. The puncturing element may be adapted to receive a guidewire through a lumen formed by the puncturing element.

The previously described systems may comprise an external imaging means for imaging the body duct of a patient in two or three dimensions, in particular the previously described imaging means, which is preferably connected to a processing unit of the system for controlling the actuation unit. The processing unit may be adapted to control the actuation unit based on the images of the external imaging means.

The previously described systems may be adapted for detecting a temporal change of a vascular pressure of a body duct. The previously described systems may comprise a cardiac cycle detector, in particular by external imaging means, preferably an ultrasound probe, for detecting a temporal change of the vascular pressure of a body duct.

The processing unit can be adapted to predict the temporal change of vascular pressure of the body duct based on the collected data of the cardiac cycle detector. The processing unit can be adapted to control the actuation unit such that the puncturing element of the guiding unit is advanced into the body duct when the predicted vascular pressure reaches a maximum, e.g. at a point in time the vessel has a maximum stiffness and a maximum cross-sectional diameter during systole. This allows the puncturing element to be inserted into the body duct more easily, minimizing the risk of missing the body duct when advancing the puncturing element.

The previously described systems may have a non-sterile part formed at least by a first transmission unit of the actuation unit, and in particular motors of the actuation unit for driving the actuation. The system may have a sterile part formed at least by the biomedical device, preferably the shearing unit, and a second transmission unit of the actuation unit. The sterile part and the non-sterile part are detachably connectable with each other such that the first and second transmission unit are functionally connected with each other for actuating a biomedical device. The sterile and/or non-sterile part may have connecting elements. The connecting elements may be protruding, recessed and/or magnetic connection elements.

In this way, the sterile part including the biomedical device can be mounted for single-use surgical procedures without the risk of compromising the sterile environment required for surgical procedures. In addition, this allows for a simple system for mounting and coupling the biomedical device such that the actuation unit can actuate at least a part of the biomedical device, e.g. the sheath, the dilator, the guiding unit and/or the shearing unit via the transmission units while retaining a sterile environment.

The protruding, recessed and/or magnetic connecting elements may simplify the connecting procedure and ensure that the connecting elements can be placed in a specific spatial orientation, e.g. that electric contacts or actuation transmission is established. If both the sterile and non-sterile part have connecting elements, the connecting elements may be correspondingly shaped with respect to each other.

The second transmission unit may comprise at least one transmission shaft, e.g. a spindle shaft, in particular a plurality of transmission shafts, for moving a mounting section for a part of the biomedical device in the longitudinal direction.

The previously described systems may have a retaining element which is adapted for retaining the sterile part and the non-sterile part in a connected state with each other. The retaining element comprises at least one connecting element, in particular a bayonet connecting element, for connecting to the sterile part and/or the non-sterile part in such a manner that the sterile part and non-sterile part are retained in the connected state.

The sterile and non-sterile part hence may remain securely connected with each other throughout actuation of the biomedical device when carrying out the percutaneous catheterization. The retaining element may have an annular shape. The retaining element may have a plurality of connecting elements, preferably bayonet connecting elements, arranged along the annular shape. The plurality of retaining elements may be adapted such that that the connecting elements, in particular the bayonet connecting elements, can be closed simultaneously, by rotation of the annular retaining element with respect to the sterile and/or non-sterile part.

The previously described systems may comprise a pressure sensor which is adapted for detecting the pressure within a cavity fluid connected or fluid connectable to a lumen of the guiding unit, in particular of the puncturing element. The pressure sensor can be arranged on the sterile part, in particular on the biomedical device.

At least one hemostasis valve may further be arranged within the biomedical device. The pressure sensor may be arranged distally from a hemostasis valve or within the hemostasis valve of the biomedical device.

The pressure sensor allows for detecting if the guiding unit was inserted into the body duct, such that the actuation of via the actuation unit can be halted once the guiding unit, in particular the puncturing element, was advanced into the body duct, preferably with a distal tip of the puncturing element arranged centrally within the body duct. A hemostasis valve may allow for reliably detecting the pressure in the lumen of the guiding unit via the pressure sensor while simultaneously introducing surgical instruments, e.g. a guidewire, or drugs through the at least one hemostasis valve into the lumen.

The pressure sensor may be connected to a processing unit of the system which may be adapted for controlling the actuation unit. The processing unit may be adapted for controlling the actuation of a guiding unit, in particular the puncturing element, by actuating the guiding unit in a longitudinal direction of the introducer assembly until detecting via the pressure sensor that the distal tip of the guiding unit was advanced into the body duct.

This allows for a secure catheterization in a partially or fully automated manner. In addition, this allows to avoid completely puncturing the body duct with the guiding unit/puncturing element through both sides of the body duct or to merely perform an insufficiently forceful, yet atraumatic, puncturing which only damages the outer vessel wall without the distal tip being endoluminally introduced.

The second transmission unit may be at least partially formed extendable parallel to the longitudinal direction. The second transmission unit may be telescopically extendable parallel to the longitudinal direction by comprising a plurality of at least partially nested transmission elements.

This allows the second transmission unit to be extended in unison with the movement of a mounting section of the system which is adapted for releasably mounting at least one part of the introducer assembly, in particular the dilator, the sheath, and the guiding unit, preferably the puncturing element, the guidewire, and/or the insertion guide.

The plurality of partially nested transmission elements may have a non-circular cross-sectional shape. The transmission elements may preferably be formed such that a rotation of a proximal most transmission element results in a rotation of a distal most transmission element.

The actuation unit may comprise a third transmission unit. The third transmission unit may be arranged at a distal end of the second transmission unit, in particular attached to a mounting section for releasably mounting at least one part of the introducer assembly. The third transmission unit may be adapted for converting a rotational movement of the second transmission unit to a movement of the dilator, the sheath, and/or the guiding unit, in particular the puncturing element, the guidewire, and/or an insertion guide, in the longitudinal direction while the third transmission unit remains translationally immovable.

In a preferred embodiment, the third transmission unit is adapted for advancing/retracting the guidewire in the longitudinal direction.

The third transmission unit may allow for actuating at least one part of the introducer assembly by a rotational movement without the third transmission unit necessarily being moved along the longitudinal direction.

The previously described systems, in particular the biomedical device, may comprise an outer guiding unit, e.g. formed similar to a trocar. The outer guiding unit may comprise a tubular member. The tubular member may have a lumen with a proximal and distal opening which has a cross-sectional dimension adapted for receiving components of the biomedical device, in particular the sheath, the dilator, and/or the puncturing element, within the outer guiding unit, in particular in a concentric manner.

The system, in particular a system comprising a shearing unit, may be adapted to advance the outer guiding unit in a longitudinal direction of the introducer assembly through an insertion site, optionally by the outer guiding unit having a sharp distal tip, e.g. via actuation by a previously described actuation unit.

The outer guiding unit may have a sealing element which is adapted for sealing the lumen, in particular at a distal end of the lumen. The sealing element may be formed by a balloon, a diaphragm, in particular a silicone diaphragm, or a check valve, in particular a ball check valve.

This allows for selectively sealing and opening the lumen of the outer guiding unit such that the outer guiding unit can be sealed when being inserted into a patient and opened when advancing components of the introducer assembly, such as a sheath.

Alternatively, the outer guiding unit may comprise an inner tubular member without a lumen, such that the inner tubular member is adapted to occlude/seal the lumen of the outer tubular member of the guiding unit.

The distal inner tubular member may have a blunt pointed end so that it protrudes from the outer tubular member, making it easier to insert the outer guiding unit. The inner tubular member may have a sharp distal tip for insertion of the outer tubular member. This may allow to remove the sharp distal tip after placement of the outer tubular member by removing the inner tubular member to reduce the risk of injury when inserting medical instruments.

The actuation unit may be adapted for moving the outer and inner tubular member independently from each other, such that the tubular members can be advanced into the patient together and the inner tubular member can be subsequently retracted without removing the outer tubular member to clear the lumen. The tubular members may be translationally movable by the actuation unit along guiding elements, in particular guiding rails, preferably in the longitudinal direction of the introducer assembly.

The outer tubular member may be more rigid than the inner tubular member, such that the inner tubular member can be removed from the outer tubular member in a curved trajectory of the inner tubular member. This allows the inner tubular member to be removed without blocking a longitudinal advancement direction of the introducer assembly, such that e.g. the sheath/dilator/puncturing element can be directly inserted into the lumen once the inner tubular member was removed in the curved trajectory.

The outer guiding unit, in particular the outer tubular member, may have a length which is sufficient such that the proximal opening can remain outside the patient and the distal opening can remain inside the patient but external from the body duct to be catheterized. In particular, the outer guiding unit may have a length between 3 cm-15 cm, in particular between 3.5 cm-11 cm, preferably between 4 cm-8 cm.

The inner tubular member may have a greater length than the outer tubular member such that it can be retracted from the lumen easily, in particular in the curved trajectory.

The outer tubular member of the outer guiding unit may have a slit, in particular two slits arranged on opposing lateral sides of the guiding unit, formed along a longitudinal direction of the guiding unit. The slit(s) may allow for easy removal of the inner tubular member. The actuation unit may be adapted for rotating the outer tubular member to align the slit such that the inner tubular member can be removed more easily.

The two slits may extend over most or the entire longitudinal length of the outer tubular member, such that the outer tubular member is or can be split by advancing components of the introducer assembly into the outer tubular member.

Two slits on opposing lateral sides further allow to advance a shearing unit synchronized/in unison with the outer guiding unit to facilitate the catheterization.

In a preferred embodiment, the previously described systems comprise a rotational actuator, in particular comprised by the actuation unit, which is adapted for rotationally moving at least one of, in particular all of the guiding unit, the dilator, and/or the sheath of the biomedical device along their angular directions.

This rotational movement facilitates insertion of the biomedical device or its components through the insertion site by reducing static friction with the tissue of the patient.

The rotational actuator may be operated via a processing unit of the system. The processing unit and the rotational actuator may be configured for performing the rotation in a continuous rotational movement. Alternatively, the processing unit and the rotational actuator may be configured for performing an oscillating rotational movement in two opposing angular directions in a defined angular range, preferably less than a full continuous rotation, in particular less than 90°.

This oscillating rotational movement also reduces the static friction and a rapid oscillating rotation transfers less unidirectional force to the tissue of rotation than a unidirectional rotation and is therefore more atraumatic.

The oscillating rotational movement may have a frequency between 5 Hz to 300 Hz, in particular 10 Hz to 200 Hz, preferably 20 Hz-100 Hz.

The processing unit and rotational actuator may be configured to have an angular range of the oscillating rotational movement between 0.01° and 90°, in particular between 0.01° and 30°, preferably between 0.01° and 10°.

According to another aspect of the invention the object is further solved by a method for percutaneous catheterization of a body duct, preferably using a previously described biomedical device with an introducer assembly. The method comprises the following steps:
Inserting an inner guiding unit through a skin of a patient into the body duct using a puncturing element,
Optionally inserting a guidewire of the inner guiding unit through the insertion guide into the body duct,
Guiding a dilator along the guiding unit without full removal of the puncturing element at a proximal end of the introducer assembly,
Advancing a sheath over the dilator into the body duct,
Optionally retracting the dilator, the inner guiding unit, in particular the insertion guide, and/or the guidewire.

The method can comprise the step of releasing the guidewire from a retaining structure of the puncturing element of the introducer assembly.

The method can comprise the step of measuring the blood

flow through the guiding unit, in particular the insertion guide, which is introduced into the body duct.

The method can comprise the step of exchanging the guidewire used for the insertion for a more flexible guidewire for navigation within the body duct.

The object is further solved by a method for aligning an external imaging means relative to a body duct, preferably using a system as previously described. The method comprises the steps of:
imaging a plane of the body duct using the external imaging means,
aligning the external imaging means in translational degrees of freedom and/or rotational degrees of freedom of the imaging means, preferably using the device positioner, such that the body duct is centred in a field of view of the imaging means and positioned along an orthogonal direction of the imaging plane or in a range of 30°, in particular 15°, relative to the orthogonal direction,
aligning the external imaging means in the translational and/or rotational degrees of freedom, preferably using the device positioner, such that the body duct remains centred in the field of view and the imaging plane is positioned at a parallel direction of a longitudinal axis of the body duct.

A longitudinal direction of the imaging plane can in addition also be positioned orthogonal to the longitudinal axis of the body duct.

The method can also comprise the step of registering data of a preoperative scan based on one other anatomical feature, in particular calcifications, to the body duct imaged by the external imaging means.

The method can further comprise the step of aligning the external imaging means in the translational and/or rotational degrees of freedom, preferably using the device positioner, such that this registered anatomical feature is avoided by the insertion path of the insertion of an introducer assembly.

### BRIEF DESCRIPTION OF THE DRAWINGS

The invention is now described with reference to certain embodiments and figures which show:

FIGS. 1A and 1B: longitudinal cross-sectional views of a first and second embodiment of a biomedical device,

FIG. 2A: a longitudinal cross-sectional view of a third embodiment of a biomedical device comprising an insertion guide,

FIG. 2B: a schematic arrangement of an actuation unit of a system comprising a biomedical device,

FIG. 2C: a longitudinal cross section of a system comprising the third embodiment of the biomedical device according to FIG. 2A and the actuation unit according to FIG. 2B,

FIGS. 3A to 3F: longitudinal cross-sectional views of the third embodiment of the biomedical device according to FIG. 2A being introduced through a skin of a patient,

FIG. 4: a longitudinal cross-sectional view of a fourth embodiment of the biomedical device comprising an insertion guide,

FIGS. 5A and 5B: schematic side views of a fifth embodiment of the biomedical device with an attachment mechanism,

FIG. 5C: a cross-sectional view of a sixth embodiment of the biomedical device with a retaining structure,

FIGS. 5D to 5F: cross-sectional views of a first and a second embodiment of a retaining structure of the puncturing element according to FIG. 5C,

FIGS. 6A to 6D: longitudinal cross-sectional views of a first and second embodiment of a dilator connected to a puncturing element comprising an actuating element in different states,

FIG. 7: a longitudinal cross-sectional view of an embodiment of the biomedical device comprising a mechanical coupling,

FIGS. 8A to 8C: longitudinal cross-sectional views of an embodiment of a puncturing element which has an anchoring member,

FIGS. 8D to 8F: longitudinal cross-sectional views of the embodiment of the puncturing element with the anchoring member according to FIG. 8A to 8C arranged within a dilator which is configured to convert the anchoring member,

FIGS. 8G and 8H: front views of a first and second embodiment of the puncturing element with the anchoring member according to FIGS. 8A to 8C,

FIGS. 9A to 9C: a patient with a biomedical device being inserted into the femoral artery,

FIG. 10: a first embodiment of a system comprising a biomedical device for insertion into the body duct of a patient which has imaging means and guidance means,

FIGS. 11A and 11B: a schematic side view of a first and second embodiment of the system comprising the biomedical device which has imaging means fixed to an introducer assembly and movable relative to an introducer assembly,

FIGS. 12A to 12C: a schematic side view of an introducer positioner with five, four, and three degrees of freedom for positioning the introducer assembly,

FIGS. 13A to 13D: a schematic side view of an embodiment of the imaging means relative to the body duct and method of positioning the imaging means relative to the body duct,

FIGS. 14A to 14C: a top view and two perspective side views of an embodiment of the system with a cutting element having a mounted biomedical device,

FIGS. 15A and 15B: a top view of the system according to FIGS. 14A to 14C and a method of advancement and retraction of the introducer assembly via the actuation unit of the system,

FIGS. 15C-15H: a perspective side view of a system for carrying out a percutaneous catheterization which has a shearing unit at a distal tip of the system for performing a sheared aperture,

FIG. 15I: a perspective view of an embodiment of the outer guiding unit which has at least one sealing member,

FIGS. 15J-15L: perspective side views of a first embodiments of a shearing unit which has a non-rotationally symmetric cross-section at a blunt distal tip and a second embodiment of a shearing unit which has two shearing elements which are radially movable with respect to each other for performing a sheared aperture,

FIG. 15J.1: a perspective view of an embodiment of the blunt inserter with a rounded distal tip,

FIGS. 15M and 15N: a schematic side view of a system for sequentially advancing a biomedical device and a shearing unit through the skin of a patient up to a body duct using a shearing unit for repeatedly performing sheared apertures,

FIGS. 15O-15R: a schematic side view of inserting and placing an outer guiding unit through an insertion site almost up to a body duct and subsequent catheterization using a biomedical device,

FIG. 16: a longitudinal cross-sectional view of an embodiment of the biomedical device comprising two mechanical couplings,

FIGS. 17A and 17B: a top view of an embodiment of the biomedical device which comprises an introducer assembly having a guidewire, a puncturing element, a dilator, a sheath, and a side port separately from each other and nested within each other respectively,

FIGS. 18A and 18B: a perspective view and a top view of an embodiment of a non-sterile part of a system for percutaneous catheterization comprising a first transmission units of the actuation unit,

FIG. 18C: a top view of an embodiment of a sterile part comprising a second transmission unit of the actuation unit,

FIG. 18D: a perspective side view of an embodiment of a retaining element adapted for retaining the sterile part of FIG. 18A and the non-sterile part of FIG. 18B in a connected state together,

FIG. 19A: a perspective side view of an embodiment of a mounting section of the dilator of a system for percutaneous catheterization,

FIGS. 19B and 19C: a perspective side view of an embodiment of a mounting section of the sheath of a system for percutaneous catheterization which has an engagement member which is convertible between an engagement configuration and a disengagement configuration,

FIGS. 20A and 20B: a perspective side view of an embodiment of an extendable part of a second transmission unit of an actuation unit of a system for percutaneous catheterization which is extendible in a longitudinal direction,

FIGS. 21A-21C: a perspective side view and a side view of an embodiment of a third transmission unit of an actuation unit of a system for percutaneous catheterization and a perspective side view of the third transmission unit of FIGS. 21A and 21B attached to a mounting section for a guiding unit,

FIG. 21D: a perspective side view of an embodiment of a sterile part 54 of the system for carrying out a percutaneous catheterization in an at least partially automated manner,

FIGS. 22A-22J: a top view of an embodiment of a system comprising the sterile part of FIG. 21D and a non-sterile part carrying out the steps of percutaneous catheterization with a biomedical device.

### DETAILED DESCRIPTION OF THE PREFERRED EMBODIMENTS

The introducer assembly 1 has an outer sheath 3, a dilator 2, and an inner guiding unit 4 arranged concentrically within each other along a longitudinal direction L of the introducer assembly 1. The sheath 3 circumferentially encloses the dilator 2 along its longitudinal length. It has a distal opening 32 at its distal end. The sheath 3 has a tapered section 31 at an outer surface neighboring the distal end and wings 33 for guiding the sheath 3 at a proximal end. The sheath 3 has a proximal opening 34 at its proximal end.

The dilator 2 extends through the distal opening 32 and the proximal opening 34 of the sheath 3.

The dilator 2 further has a distal opening 22 at its distal end. The distal end has a tapered outer section 21 for insertion through a skin and tissue into a body duct of a patient over the guiding unit 4. The dilator further has a proximal opening 25 at its proximal end. The dilator 2 circumferentially encloses the guiding unit 4 along its length.

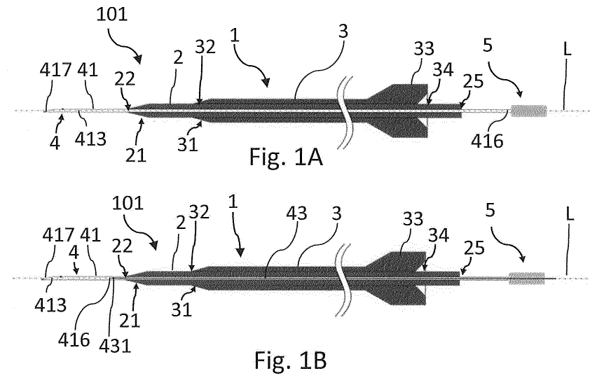

The guiding unit 4 in FIG. 1A and 1B protrudes from the first and second opening 22, 25 of the dilator 2. The sheath 3, the dilator 2, and the guiding unit 4 are all movable relative to each other in the longitudinal direction of the introducer assembly 1.

The guiding unit 4 is FIG. 1A comprises a puncturing element 41 formed by a continuous stainless-steel cannula which circumferentially encloses a cylindrical lumen 413 and has a puncturing section 417 at its distal end. The lumen 413 extends from a distal opening formed by the puncturing section 417 to a proximal end 416 at a proximal end 5 of the introducer assembly 1.

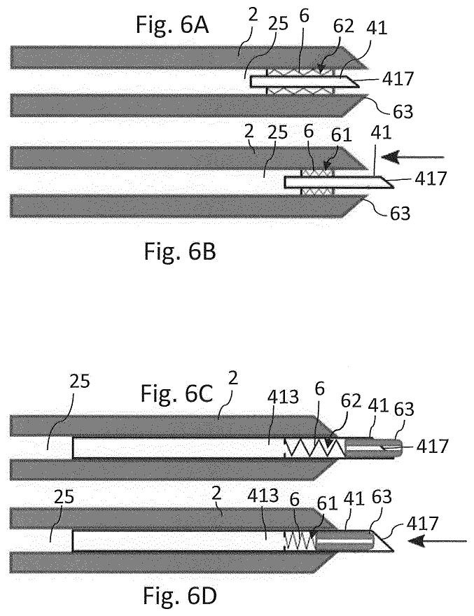

The cannula 41 can have a length in a range from 6 cm to 35 cm, in particular 15 cm. The cannula 41 in FIG. 1A is sufficiently stiff, such that the tapered outer section 21 of the dilator 2 can be introduced along the cannula 41 without requiring a separate guidewire. The cannula 41 can optionally be a Veress cannula (see FIG. 6B) for shielding the puncturing section 417 to protect a tissue wall of the body duct of the patient during introduction of the dilator 2 and/or the sheath 3.

The guiding unit 4 in FIG. 1B comprises a puncturing element formed by a continuous cylindrical stainless-steel cannula 41 and an additional guidewire 43. The cannula 41 has a shorter length than in FIG. 1A in a range from 0.1 cm to 6 cm, in particular 0.5 cm. The cannula 41 has a distal opening formed by the puncturing section 417 at its distal end and circumferentially encloses a cylindrical lumen 413. A proximal end 416 of the cannula 41 forms a smooth transition with a distal end 431 of the guidewire 43. The cannula 41 connected to the guidewire 43 can thus be moved back and forth relative to the remaining introducer assembly 1 by the guidewire 43 acting as a kind of push rod. The guidewire 43 has a lower bending stiffness than the cannula 41, so that it can more easily follow the turns in the introducer assembly 1 and/or the body duct. The guidewire 43 is sufficiently stiff, such that the tapered outer section 21 of the dilator 2 can be introduced into the body duct along the guidewire 43.

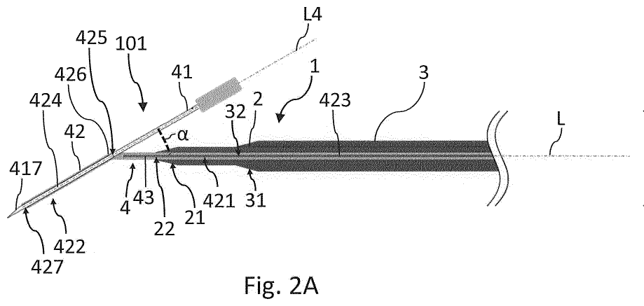

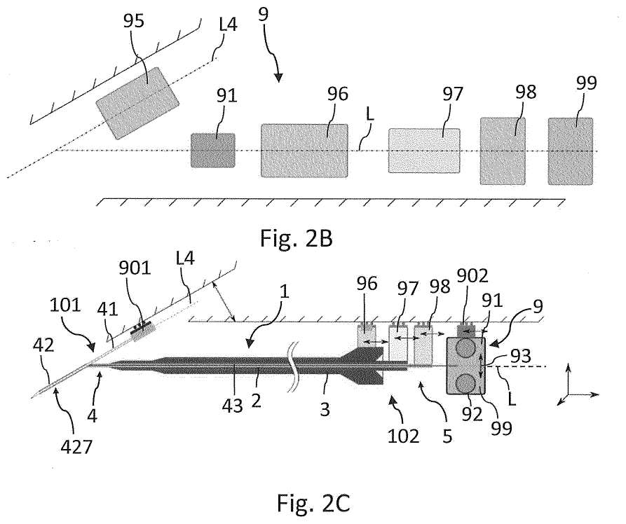

FIG. 2A shows a longitudinal cross-sectional view of a third embodiment of a biomedical device 101 comprising an introducer assembly 1 with an insertion guide 42. The introducer assembly 1 comprises a sheath 3, a dilator 2, and a guiding unit 4 which have sections which are arranged coaxially to each other along a longitudinal direction L of the introducer assembly 1. The sheath 3 has a distal opening 32 and a tapered outer section 31 on an outer surface near its distal end. The dilator 2 also has a distal opening 22 and a tapered outer section 21 on an outer surface near its distal end.

The insertion guide 42 of the guiding unit 4 has a proximal section 421 and a distal section 422. The proximal section 421 of the insertion guide 42 is circumferentially enclosed by the dilator 2 and has a proximal lumen 423 inside which a guidewire 43 is arranged. The distal section 422 of the insertion guide 42 has a distal lumen 424 which has a distal opening 427 at its distal end and a lateral opening 425 at its proximal end 426.

The distal lumen 424 extends in a straight line between the openings 425, 427. A puncturing element formed by a cannula 41 is arranged within the lumen 424 and is movable relative to the insertion guide 42 in the longitudinal direction L4 of the cannula 41.

The cannula can be a commercially available standardized stainless-steel cannula 41 which is not necessarily a part of the device. The distal section 422 is arranged at an angle α of 30° relative to the proximal section 421, such that the cannula 41 can be introduced and retracted from the lateral opening 425 of the distal section 422 along the longitudinal direction L4 for introduction of the guidewire 43.

Therefore, the step of introducing the guidewire 43 can be carried out faster, since the cannula 41 only needs to be retracted from the lateral opening 425 over a short distance, such that the guidewire 43 can be introduced into the body duct. The cannula 41 is not arranged concentrically around the guidewire 43, such that the cannula 41 does not have to be removed from a proximal end of the introducer assembly 1 after the insertion of the cannula 41.

The insertion guide 42 can be introduced through a skin and tissue of a patient in parallel with the cannula 41 with a puncturing section 417 which protrudes from the distal opening 427 or be advanced around the cannula 41 was introduced into the body duct. The distal opening 427 of the insertion guide 42 instead forms a blunt edge such that the insertion guide 42 is configured not to damage a tissue wall of the body duct.

FIG. 2B shows a schematic arrangement of an actuation unit 9 of a system comprising a biomedical device as described above. The actuation unit 9 has a clutch 91 which is switchable between different configurations 95, 96, 97, 98, 99 for grasping one of the components of the introducer assembly 1. In a first configuration 95 the actuation unit 9 or an additional actuation unit 9 is configured for grasping a cannula and moving the cannula relative to the remaining introducer assembly in a longitudinal direction L4.

In a second configuration 96, the actuation unit 9 is configured for grasping the sheath.

In a third configuration 97 the actuation unit 9 is configured for grasping the dilator.

In a fourth configuration 98 the actuation unit 9 is configured for grasping the insertion guide, and in a fifth configuration 99 for grasping the guidewire.

In one of these configurations 96, 97, 98, 99 the clutch 91 can move the respective grasped component of the introducer assembly in a longitudinal direction L relative to the remaining introducer assembly.

Alternatively to the actuation unit 9 of FIG. 2B at least some, in particular all, of the different components of the introducer assembly 1 may have an individual actuation unit which is configured for only actuating the according component in a longitudinal direction L, L4.

FIG. 2C shows a longitudinal cross-sectional view of a system 102 comprising the third embodiment of the biomedical device 101 according to FIG. 2A and the actuation unit 9 according to FIG. 2B. The actuation unit 9 has a clutch 91 which comprises two rollers 92 which are adjustable to multiple different interdistances 93 and are arranged radially outward on two opposing sides of the introducer assembly 1.

The interdistance 93 between the rollers 92 in this embodiment is essentially arranged orthogonal to a longitudinal direction L of the insertion assembly 1. The sheath 3, dilator 2, and an insertion guide 42 and a guidewire 43 of the guiding unit 4 have at least partially exposed sections which are spaced from each other at a proximal end 5 of the insertion assembly 1. The actuation unit 9 comprises a longitudinal actuator 902 such that the clutch 91 can be moved in the longitudinal direction L of the introducer assembly 1, preferably along a rail parallel to the introducer assembly 1.

The longitudinal actuator 902 can be used to move the clutch to the partially exposed sections of the sheath 3, dilator 2, the insertion guide 42, and the guidewire 43. The interdistance 93 of the rollers 92 can then be adjusted to engage the partially exposed section. Thereby, a configuration 96, 97, 98, and 99 of the actuation unit 9 can be chosen to actuate the sheath 3, the dilator 2, the insertion guide 42, or the guidewire 43 in the longitudinal direction L by rotating the rollers 92 which frictionally engage the respective exposed section.

In FIG. 2C, a cannula 41 arranged within the distal section 427 of the insertion guide 42 is movable by a second actuation unit 901 along a longitudinal direction L4 at an angle relative to the longitudinal direction L of the introducer assembly 1.

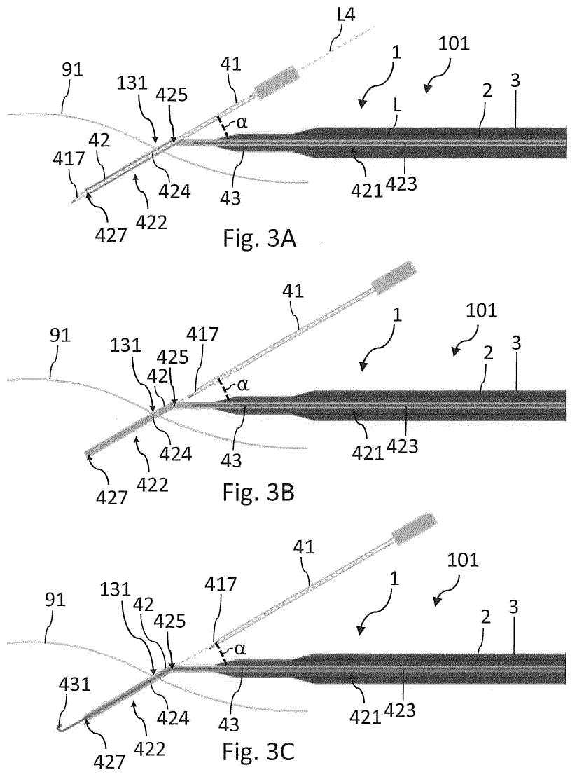

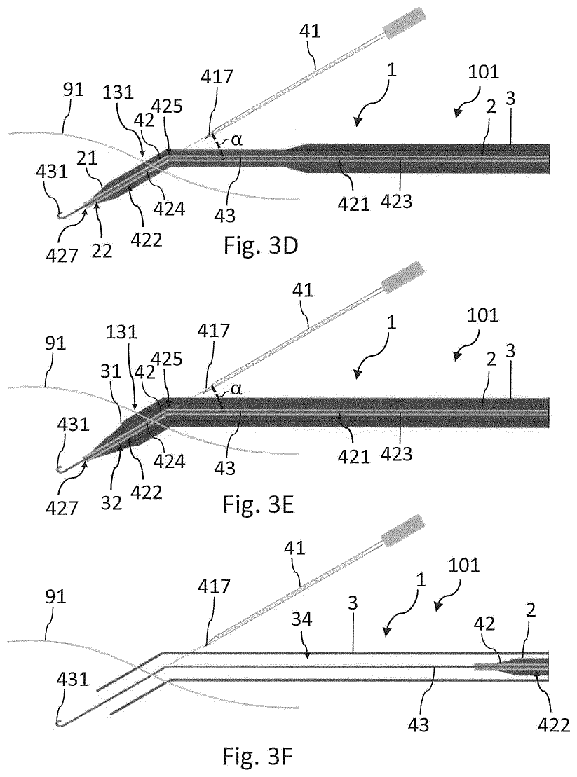

FIGS. 3A to 3F show longitudinal cross-sectional views of the third embodiment of the biomedical device 101 according to FIG. 2A introduced in several steps through a skin 91 of a patient at an insertion site 131.

In FIG. 3A, the cannula 41 is arranged within the distal lumen 424 of the distal section 422 of the insertion guide 42.

The insertion guide 42 with the puncturing section 417 of the cannula 41 protruding from the distal opening 427 is introduced through the skin 91 in the longitudinal direction L4 of the cannula 41. The longitudinal direction L4 is arranged at the angle α of 30° relative to the longitudinal direction L (see FIG. 2A and FIG. 2C). The distal section 422 has a longitudinal length such that it is insertable into the body duct with the distal opening 427 and at the same time the lateral opening 425 of the distal section 422 is disposed outside the patient's skin 91. The longitudinal direction L4 of the distal section 422 can be arranged at a chosen angle with respect to a longitudinal axis of the body duct.

The guidewire 43 is arranged within the proximal section 423 of the insertion guide 42. The proximal section 423 is circumferentially surrounded by the dilator 2 and the sheath 3. An inner diameter of the dilator 2 corresponds to the outer diameter of the proximal section 421 and distal section 422 of the insertion guide 42.

FIG. 3B shows that the cannula 41 is retracted from the lateral opening 425 of the distal section 422, such that the distal lumen 424 is free from obstacles for inserting the guidewire 43.

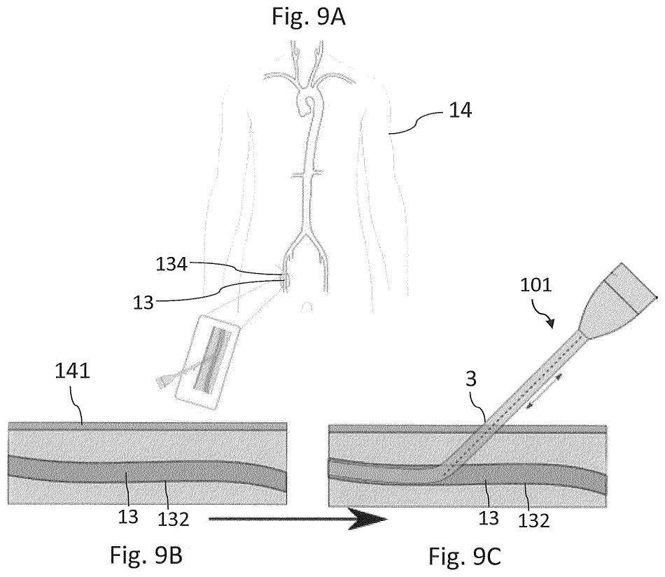

FIG. 3C shows that a distal end 431 of the guidewire 43 is introduced through the distal lumen 424 of the insertion guide 42 into the body duct (see FIGS. 9B and 9C) of the patient.

FIG. 3D shows that the dilator 2 is introduced through the skin 91 of the patient with the tapered outer section 21 at its distal end. For this purpose, the distal opening 22 of the dilator is advanced over the insertion guide 42. The tapered outer section 21 of the dilator widens the insertion site 131, such that the dilator can be advanced.

FIG. 2E shows that the sheath 3 with the tapered outer section 31 is introduced through the skin of the patient with the distal opening 32 being advanced over the dilator 2 into the body duct.

FIG. 3F shows that the dilator 2 and the insertion guide 42 are retracted from a lumen 34 of the sheath 3, while the guide wire 43 stays in place, for performing a surgical procedure.

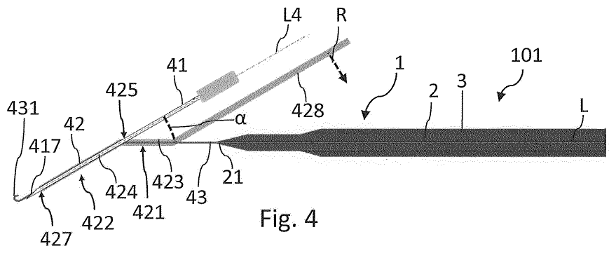

FIG. 4 shows a longitudinal cross-sectional view of a fourth embodiment of a biomedical device 101 comprising and insertion assembly 1 which has an insertion guide 42. The device 101 is largely analogous to the device of FIG. 2A and reference is made to features which were previously described. However, in contrast to FIG. 2A the biomedical device 101 in FIG. 4 has a dilator 2 which is arranged coaxially enclosing a guidewire 43 instead of the insertion guide 42. An inner diameter of a lumen of the dilator 2 thus corresponds to the diameter of the guidewire 43. Therefore, a sheath 3, the dilator 2, and the guidewire 43 can be commercially available components or a set for performing the Seldinger technique. Apart from that the guidewire 43 can be introduced into a body duct through the insertion assembly 1 analogous to FIGS. 3A to 3F.

The insertion guide 42 can be inserted using a puncturing surface 417 of a cannula 41 protruding from the distal opening 427 of the insertion guide 42. The cannula 41 can be advanced and retracted from the distal section 422 of the insertion guide 42 at an angle α relative to a longitudinal direction L of the introducer assembly 1. After retraction of the cannula 41 in the longitudinal direction L4 of the cannula 41, the guidewire 43 can be advanced into a body duct from the distal opening 427.

FIG. 4 shows a mantle section 428 of the insertion guide 42 being partially open in a circumferential area of the insertion guide 42, such that the guidewire 43 can be released from a proximal and a distal lumen 423, 424 of a proximal and the distal section 421, 422 in a radial direction R of the insertion guide 42. The partially open circumferential area extends over the entire length of the insertion guide 42.

Thereby, the insertion guide 42 can be separated from the guidewire 43 by retracting the insertion guide 42 relative to the guidewire 43, such that the guidewire 43 is radially released through the opening of the mantle surface 428. The insertion guide 42 is retracted along an additional translational path L3. Retracting the insertion guide 42 does not move the guidewire 43, such that a distal end 431 of the guidewire 43 can be retained in a body duct.

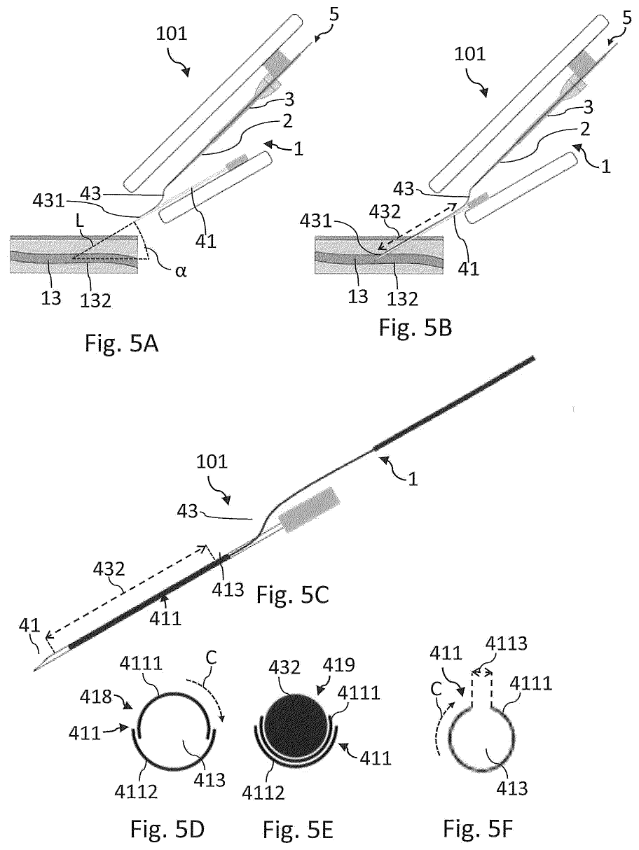

FIGS. 5A and 5B show schematic side views of a fifth embodiment of the biomedical device 101 comprising an introducer assembly 1 which has a guidewire 43 attached to a distal end 431 of a puncturing element 41. The distal end 431 comprises a spring-loaded attachment mechanism which attaches the guidewire 43 to the distal end 431, such that a longitudinal portion 432 of the guidewire 43 extends essentially parallel to a longitudinal direction L of the puncturing element 41. However, the guidewire 43 is arranged outside of a lumen of the puncturing element 41. The spring-loaded attachment mechanism is configured to retain the guidewire 43 during the insertion of the puncturing element 41 and to release the guidewire when passing through a tissue wall 132 of a body duct 13. Thereby, the guidewire 43 can be introduced into the body duct 13 without having to remove the puncturing element 41 at a proximal end 5 of the introducer assembly 1. A dilator 2 and a sheath 3 can be directly introduced into the body duct 13 subsequently or in parallel to the removal of the puncturing element 41.

FIG. 5C shows a cross-sectional view of a sixth embodiment of the biomedical device 101 with an introducer assembly 1 which has a puncturing element 41 which has a retaining structure 411 (see FIG. 5D and FIG. 5E) that circumferentially encloses a guidewire 43 at least partially in a lumen 413 along a longitudinal portion 432 of the guidewire 43. The guidewire 43 in FIG. 5C is pre-loaded within the lumen 413.

FIGS. 5D and 5E show a first embodiment of the retaining structure 411 of the puncturing element 41 according to FIG. 5C. The retaining structure 411 in FIG. 5D has a first C-shaped mantle surface 4111 and a second C-shaped mantle surface 4112 which enclose the lumen 413 of the puncturing element 41 in a retaining state 418, such that the longitudinal portion 432 of the guidewire 43 (see FIG. 5C) can be retained in the lumen 413. The C-shaped mantle surfaces 4111, 4112 are rotatable relative with respect to each other in a circumferential direction C of the puncturing element 41 to convert the retaining structure 411 from the retaining state 418 to a radially open releasing state 419. The C-shaped mantle surfaces 4111, 4112 have different diameters such that the outer mantle surface 4112 in FIG. 4E encloses the inner mantle surface 4111. Thus, in a releasing state 419, the longitudinal portion 432 of the guidewire 43 can be released radially outwardly from the puncturing element 41.

FIG. 5F shows a second embodiment of the retaining structure 411 of the puncturing element 41 according to FIG. 5C. The retaining structure 411 has a single mantle surface 4111 which only partially encloses the lumen 413 of the puncturing element 41 in a circumferential direction C of the puncturing element 41. The diameter of the guidewire can be larger or approximately the same as a circumferential size 4113 of a slot in the mantle surface (4111). The longitudinal portion of the guidewire 43 of FIG. 5C can be released radially outwardly from the lumen 413 by a applying a predetermined amount of force when retracting the puncturing element 41 relative to the guidewire.

FIGS. 6A to 6D show longitudinal cross-sectional views of a first and second embodiment of a dilator 2 connected to a puncturing element 41 comprising an actuating element 6. The actuating element 6 is formed by a spring which is convertible between a compressed state 61 in FIG. 6B and FIG. 6D and an extended state 62 in FIG. 6A and FIG. 6C.

FIGS. 6A and 6B show the puncturing element in the form of a cannula tip 41 being connected to the dilator 2 via the actuating element 6. The cannula tip 41 is arranged inside a lumen 25 formed by the dilator 2. In the expanded state 62 of the actuating element 6 a puncturing surface 417 of the cannula tip 41 is retracted behind a non-puncturing surface 63 formed by a distal end of the dilator 2. In the compressed state 61 of the actuating element 6 the puncturing surface 417 is advanced in a longitudinal direction of the dilator 2 such that the puncturing section 417 protrudes from the non-puncturing surface 63 of the dilator 2.

The FIGS. 6C and 6D show the puncturing element in the form of a cannula tip 41 being directly connected to the dilator 2 within the inner lumen 25 of the dilator 2. The actuating element 6 is arranged within an inner lumen 413 of the cannula tip 41. In FIG. 6C the spring of the actuating element 6 is in an expanded state 61 such that a non-puncturing surface 63 located concentrically within the cannula is advanced forward to shield a puncturing section 417 of the cannula tip 41 analogously to a Veress cannula. Thereby, the cannula tip 41 cannot injure a wall of a body duct in the expanded state 61. In the retracted state 61 of the spring in FIG. 6D, the non-puncturing surface 63 located within the lumen 413 of the cannula tip 41 is retracted behind the puncturing surface 417, such that the cannula tip 41 can be introduced through a skin of a patient into the body duct.

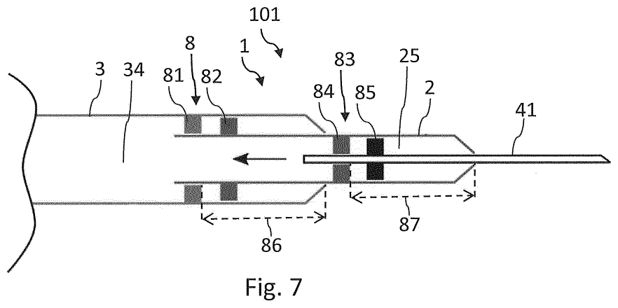

FIG. 7 shows a longitudinal cross-sectional view of an embodiment of the biomedical device 101 comprising an introducer assembly 1 which has a sheath 3, a dilator 2, a cannula tip 41, and a first and a second mechanical coupling 8, 83. Such mechanical couplings 8, 83 can be arranged in any of the aforementioned first to fifth embodiments of the biomedical device 101.

The first coupling 8 has a first coupling element 81 formed by a radially inwardly projecting annular protrusion of the sheath 3 and a second coupling element 82 formed by a radially outwardly projecting annular protrusion of the dilator 2. Thereby, only a predetermined section 86 of the dilator 2 is insertable into a lumen 34 of the sheath 3 before the coupling elements 81, 82 block each other. The coupling elements 81, 82 can also be arranged the other way around such that only a predetermined section 86 of the dilator 2 can be introduced from the lumen 34.

The second coupling 83 has a first coupling element 84 formed by a radially inwardly projecting annular protrusion of the dilator 2 and a second coupling element 85 formed by a radially outwardly projecting annular protrusion of the cannula tip 41. Thereby, only a predetermined section 87 of the dilator 2 is insertable into a lumen 25 of the dilator 2 before the coupling elements 84, 85 block each other. The coupling elements 84, 85 can also be arranged the other way around such that only a predetermined section 87 of the puncturing element 41 can be introduced from the lumen 25.

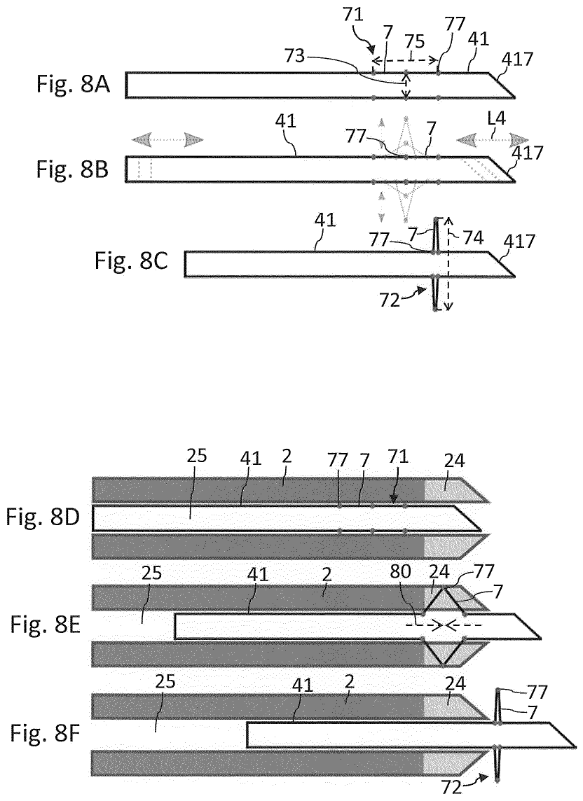

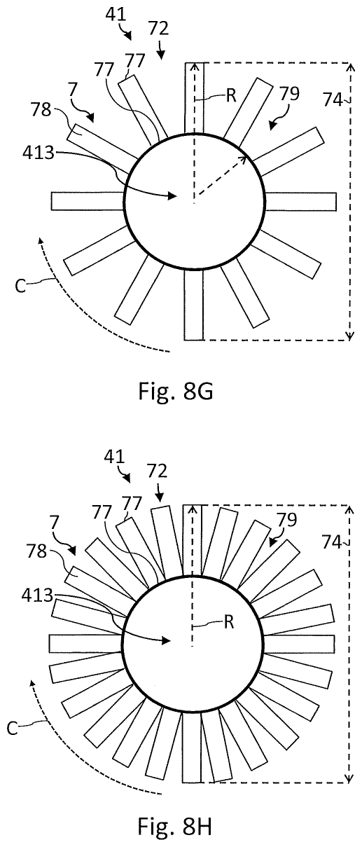

FIGS. 8A to 8C show longitudinal cross-sectional views of an embodiment of a puncturing element 41 which has an anchoring member 7. The anchoring member 7 is formed by a longitudinal section 75 of the puncturing element 41 and is arranged partially circumferentially around the puncturing element 41. FIG. 8A shows the anchoring member 7 in a collapsed configuration 71 which has a collapsed radial dimension 73. The anchoring member 7 is formed by three longitudinally spaced circumferential rows of joints 77 which allow the anchoring member 7 to be converted from the collapsed configuration 71 to an expanded configuration 72 (FIG. 8B). In the expanded configuration 72 in FIG. 8C, the anchoring member 7 forms a star-shape which has prongs formed by the center joints 77 located at the outermost radial tips of the anchoring member 7 (see FIG. 8G and FIG. 8H). Between the prongs of the anchoring member 7, the anchoring member 7 has longitudinal slots which essentially have a diameter of the collapsed radial dimension 73.

FIG. 8B further shows that the anchoring member 7 is biased to be converted from the collapsed configuration 71 to an expanded configuration 72. The anchoring member 7 is automatically converted by a restoring spring force of a spring element (not shown in FIG. 8A to FIG. 8F) of the anchoring member 7 in a longitudinal direction L4 of the puncturing element 41. The anchoring member 7 is only retained within the collapsed configuration 71 by a static retainment within the dilator 2 (see FIG. 8D).

The anchoring member 71 can be converted to the collapsed configuration 71 from the default expanded configuration 72 by advancing the dilator 2 over the anchoring member 7 (see FIG. 8D to 8F). The joints 77 of the anchoring member 7 allow for a radial expansion due to a longitudinal compression 80 based on the restoring spring force.

FIG. 8C shows the anchoring member 7 in the fully expanded configuration 72 having a maximum expanded radial dimension 74.

FIGS. 8D to 8F show longitudinal cross-sectional views of an embodiment of the puncturing element 41 with the anchoring member 7 according to FIG. 8A to FIG. 8C arranged within a lumen 25 of a dilator 2. The dilator 2 has longitudinal slots 24 at its distal end and is configured for converting the anchoring member 7 from a collapsed configuration 71 to an expanded configuration 72 and vice versa. The longitudinal slots 24 allow the dilator 2 to be advanced over prongs 78 of the anchoring member 7 to convert the anchoring member 7 from the default expanded configuration 72 to the collapsed configuration 71 (see FIG. 8G and FIG. 8H).

The anchoring member 7 has an anchor actuator (not shown in FIG. 8D to 8F), such that the anchoring member 7 can be converted from the collapsed configuration 71 in FIG. 8D to the expanded configuration 72 in FIG. 8F by retracting the dilator 2 over the puncturing element 41 or advancing the puncturing element 41 relative to the dilator 2. The anchor actuator of the dilator 2 can engage the anchoring member 7, such that advancing the slots 24 over the anchoring member 7 can convert the anchoring member 72 from the expanded configuration 72 to the collapsed configuration 71 by applying a force directed outward in the longitudinal direction.

FIGS. 8G and 8H show front views of a first and a second embodiment of the puncturing element 41 with the anchoring member 7 of FIGS. 8A to 8C in the expanded configuration 72. The anchoring member 7 in the expanded configuration 72 comprises prongs 78 which are formed by the longitudinal section 75 (see FIG. 8A) and have an expanded radial dimension 74. Each prong 78 is formed by joints 77 which are arranged on the distal tips of the prongs 77 and on the distal and proximal ends of the longitudinal section 75 (see FIGS. 8A to 8C). Between neighboring prongs 78 the puncturing element 41 forms slits 79 in a longitudinal direction of the puncturing element 41, such that the longitudinal slots 24 of the dilator 2 (see FIGS. 8D to 8F) can be advanced over the prongs 78. The slits 79 have a smaller width in a radial direction R of the puncturing element 41 than the width of the prongs 78 in the radial direction of the anchoring member 7. The slits 79 in FIG. 8G and 8H have essentially the same radial dimension as an inner lumen 413 of the puncturing element 41.

The prongs 77 of the anchoring means 7 can be arranged spaced apart from each other or directly adjacent to each other along a circumferential direction C of the puncturing element 41 as shown in FIG. 8G and in FIG. 8H.

FIG. 9A shows a patient 14 and body ducts 13 of the patient 14 in which a biomedical device 101 is introducible. The section of a femoral artery 134 of the patient 14 into which a biomedical device 101 is inserted is shown in an enlarged view in FIG. 9B and FIG. 9C.

FIG. 9B shows the body duct 13 of the femoral artery 134 enclosed by a tissue wall 132 and a skin 141 of the patient 14.

FIG. 9C shows the sheath 3 of the device 101 introduced into the body duct 13, such that the catheterization is complete, and a subsequent medical procedure can be performed.

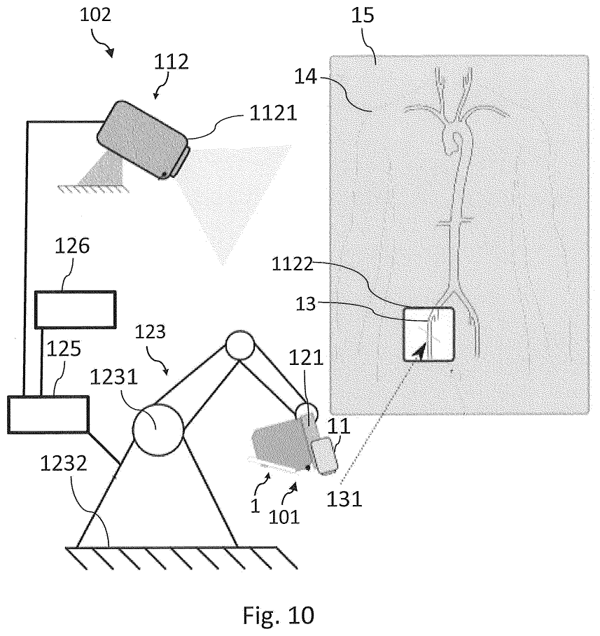

FIG. 10 shows a schematic first embodiment of a system 102 comprising a biomedical device 101 for insertion into a body duct 13 of a patient 14. The biomedical device 101 has an ultrasound probe 11 as an imaging means. The system 102 has a camera 1121 as guidance means 112. The device 101 comprises the ultrasound probe 11 and the introducer assembly 1 connected via a base plate as a common support 121. The device 101 is arranged connected to a robotic arm 123 of the system 102.

The system 102 further has a processing unit 125 which is operatively connected to an actuator of the robotic arm 123, the imaging means 11, and the camera 1121. The processing unit 125 is configured to operate the robotic arm 123 based on the images provided by the ultrasound probe 11 and the camera 1121.

The processing unit 125 is further connected to a memory unit 126 which is configured to save images of the ultrasound probe 11 and the camera 1121 and processed data of the processing unit 125 based on these images. In addition, preoperative CT data of the body duct 13 are stored on the storage unit 126, such that the processing unit 125 can register the images provided by the ultrasound probe 11 to the CT data.

The robotic arm 123 is positioned on a stationary surface 1232 and has multiple joints 1231 such that the device 101 can be positioned relative to the body duct 13 at an insertion site 131.

The camera 1121 supplies images to the processing unit 125. The processing unit 125 is configured to visually detect a landmark 1122 in the form of a known geometric shape, in particular a rectangular shape, within the supplied images. The landmark 1122 is placed on a drape 15 and frames the target site 131 for catheterization of the body duct 13, such that the target site 13 can be identified from the processing unit 125 based on the landmark 1122. The drape 15 covers the patient 4 except for the insertion site 131 to reduce unnecessary visual input for the camera 1121. Based on an input of the processing unit 125, the robotic arm 123 can position the device 101, such that the ultrasound probe 11 can be placed adjacent the body duct 13. For this purpose, the robotic arm 123 has three translational degrees of freedom t and two rotational degrees of freedom f (see FIGS. 13A and 13B). Furthermore, the robotic arm 123 is configured for rotating the device 101 in a circumferential direction of the ultrasound probe 11 (see FIGS. 13C to 13D).

The processing unit 125 is further configured to detect calcifications within the body duct 13. Based on the images provided to the processing unit 125 by the ultrasound probe 11 and a preoperative CT scan, the position of the device 101 can be adjusted using the robotic arm 123 to a different position if a predicted introduction path would pass through the detected calcifications.

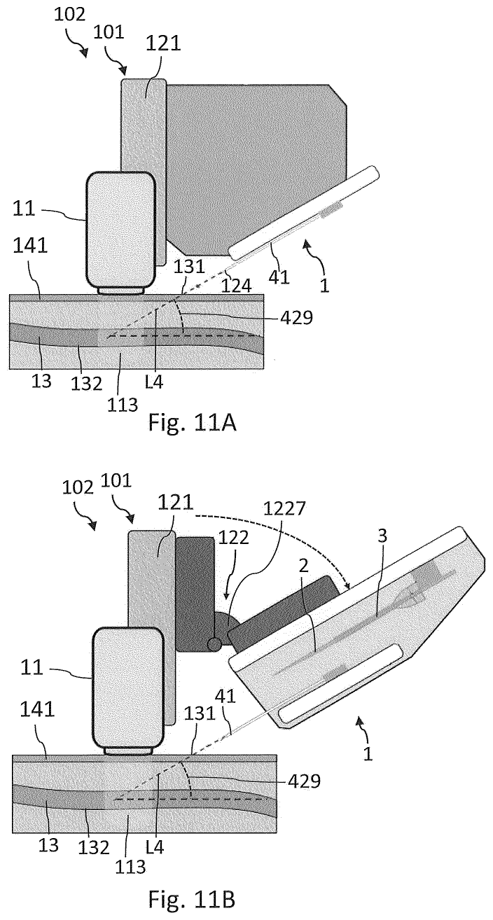

FIGS. 11A and 11B show schematic side views of a first and second embodiment of a system 102 comprising the biomedical device 101 which has an ultrasound probe 11 and an introducer assembly 1 fixedly connected to a baseplate 121. In FIGS. 11A and 11B a longitudinal axis of the ultrasound probe 11 is positioned essentially orthogonal to a longitudinal axis of the body duct (see FIGS. 13A to 13D). A field of view 113 of the ultrasound transducer is located at a section of the body duct 13 in which a puncturing element 41 of the introducer assembly 1 is introducible into the body duct 13 through an insertion site 131, a skin 141 of a patient, and a tissue wall 132 of the body duct 13.

In FIG. 11A, an angle α between a longitudinal direction L4 of the puncturing element 41 and the longitudinal direction of the body duct is fixed at 30°.

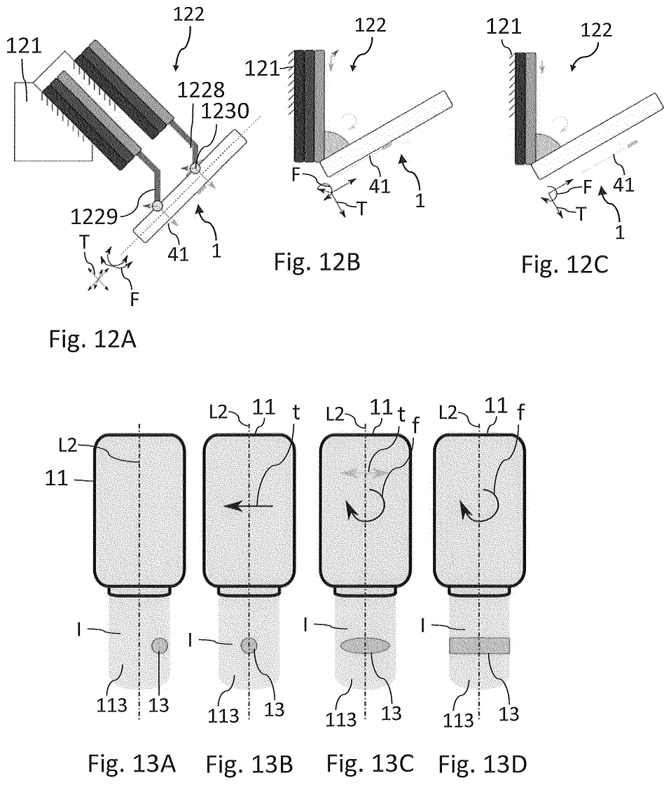

In FIG. 11B the angle α is adjustable by an electric motor 1227 of an introducer positioner 122 (see FIGS. 12B to 12C). Positioning the introducer assembly 1 and in particular adjusting the longitudinal direction L4 of the puncturing element 41 does not move the transducer 11. A dilator 2 and a sheath 3 of the introducer assembly 1 can be introduced into the body duct 13 analogously as previously described in FIG. 1A.

FIGS. 12A to 12C show a schematic side view of an introducer positioner 122 with five (FIG. 12A), four (FIG. 12B), and three (FIG. 12C) degrees of freedom T, F for positioning the introducer assembly 1. The introducer positioner 122 is fixedly connected to a common support 121 which connects the introducer positioner 122 to the imaging means (see FIGS. 11A and 11B).

FIG. 12A shows an introducer positioner 122 with three translational degrees of freedom T and two rotational degrees of freedom F of the introducer assembly 1 relative to the common support 121. The arms 1228, 1229 each have a joint 1230 at an end connected to the introducer assembly 1, such that the introducer assembly 1 is rotatable relative to the common support 121. The introducer assembly 1 with a puncturing element 41 has the three translational degrees of freedom T and two rotational degrees of freedom F.

FIG. 12B shows an introducer positioner 122 which has three translational degrees of freedom T and one rotational degree of freedom F.

FIG. 12C shows an introducer position 122 which is essentially analogous to the introducer positioner 122 of FIG. 12B but the introducer assembly 1 in FIG. 12C only has two instead of three translational degrees of freedom T.

FIGS. 13A to 13D show schematic side views of an embodiment of an ultrasound probe 11 relative to a body duct 13 and a method of positioning the ultrasound probe 11 relative to the body duct 13. In FIG. 13A the ultrasound probe 11 is placed to image the body duct 13 by the device positioner 123 (see FIG. 10). The ultrasound probe 11 is preferably configured to retrieve three-dimensional spatial information with a minimum delay at more than 10 frames per second with a total data delay of less than 100 ms.

In a first step in FIG. 13A, a field of view 113 of the ultrasound probe 11 is roughly aligned to the body duct 13, such that the field of view 113 captures a cross-section of the body duct 13 within an imaging plane I of the ultrasound probe 11.

In a second step in FIG. 13B, the ultrasound probe 11 is aligned, such that the cross-section of the body duct 13 is centered in the field of view 113 within the imaging plane I. Therefore, the ultrasound probe 11 is adjusted in at least one translational degree of freedom t by the device positioner, such that the imaging plane I is essentially orthogonal relative to a longitudinal direction of the body duct 13. Alternatively, the imaging plane I can be arranged such that the cross-section of the body duct 13 is at least fully contained within the field of view 113 of the ultrasound probe 11.

In a third step in FIG. 13C, the ultrasound probe 11 is adjusted in translational degrees of freedom t and rotational degree of freedom f until the longitudinal direction of the body duct 13 is essentially parallel to the imaging plane I as shown in FIG. 13D. In FIG. 13D the longitudinal direction of the body duct 13 is aligned essentially orthogonal to the longitudinal direction L2 of the ultrasound probe 11. During the alignment according to FIG. 13C, the translational and/or rotational degree of freedom f, t is continuously adjusted, such that the body duct 13 remains essentially centered in the field of view 113.

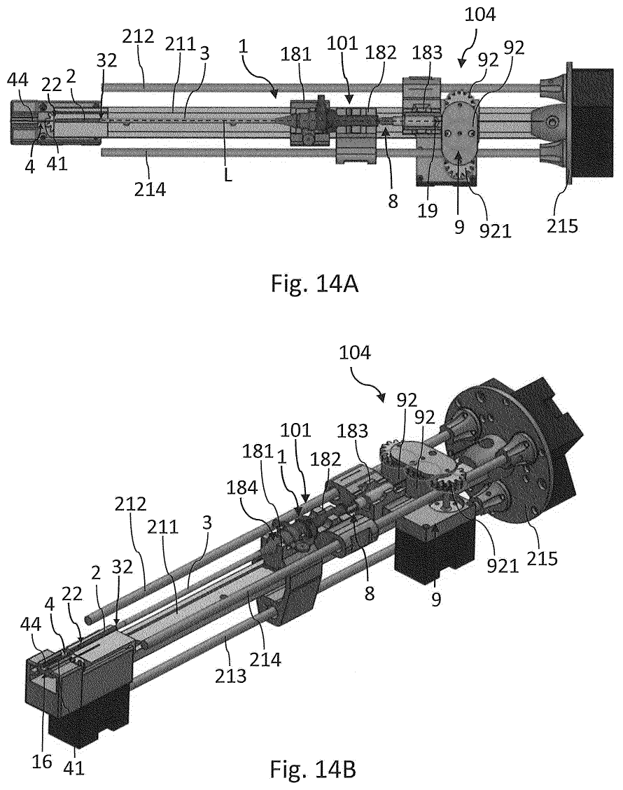

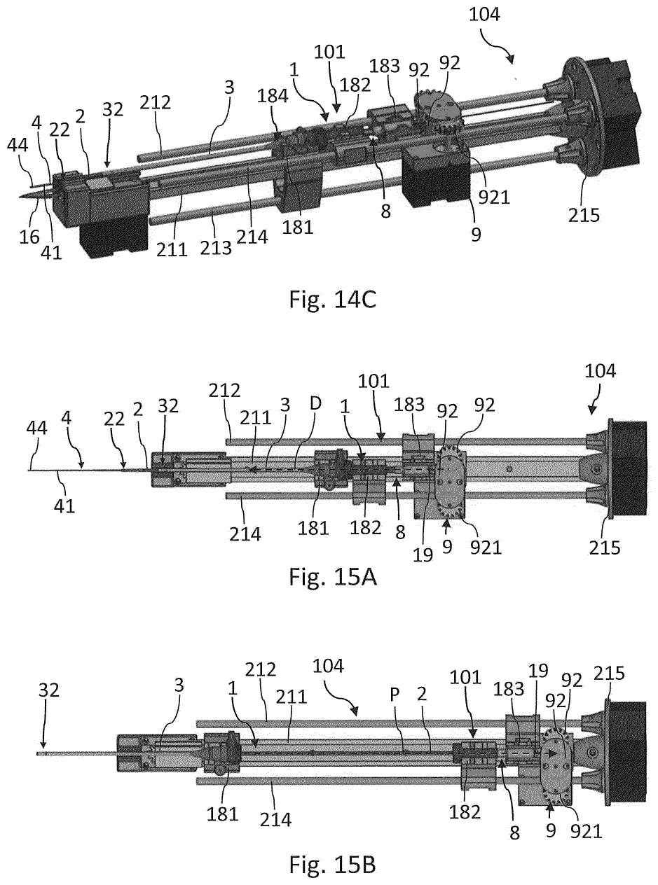

FIGS. 14A to 14C show a top view and two perspective side views of an embodiment of the system 104 with a shearing unit comprising a cutting element 16 (see FIG. 14B), formed by a scalpel, and a mounted biomedical device 101. The biomedical device 101 has an introducer assembly 1 which comprises a dilator 2, a sheath 3, and an inner guiding unit 4 which is formed by a puncturing element 41.

The system 104 has an actuation unit 9 with two rollers 92 which are driven by a gearbox for advancement and retraction of the introducer assembly 1, namely the dilator 2, the sheath 3, and the guiding unit 4. The top of the actuation unit 9 is shown as semi-opaque for better visibility of the rollers 92.

The system 104 has three mounting sections 181, 182, 183 which have an open shape 184 formed to receive a proximal section of a sheath 3, a dilator 2, and a guiding unit 4 of the biomedical device 101, respectively. The sheath 3, the dilator 2, and the guiding unit 4 can be mounted in the respective mounting sections 181, 182, 183 which form a snap-fit mechanism, such that the sheath 3, the dilator 2, and the guiding unit 4 can be arranged in a specific predetermined position with respect to each other.

The system 104 further has four guiding elements 211, 212, 213, 214 formed by metal rails which are arranged parallel to a longitudinal direction L of the system 104. A first guiding element 211 is arranged centrally between the other three guiding elements 212, 213, 214. The other three guiding elements 212, 213, 214 are each connected to a base 215 at a proximal side of the system 104 while a distal side forms a free portion.

The three mounting sections 181, 182, 183 are all movably attached to the centrally arranged guiding element 211 and one of the other three guiding elements 212, 213, 214, respectively. The three mounting sections 181, 182, 183 are movable along the guiding elements 211, 212, 213, 214 via the actuation unit 9.

The rollers 92 are driven by gears engaging in a common driven gearbox 921 of the actuation unit 9, so that both rollers 92 can perform a rotary motion at the same speed and power.

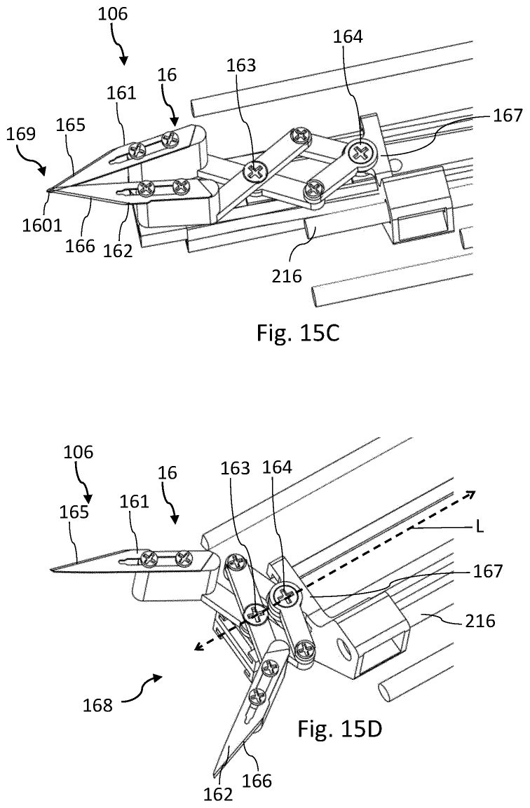

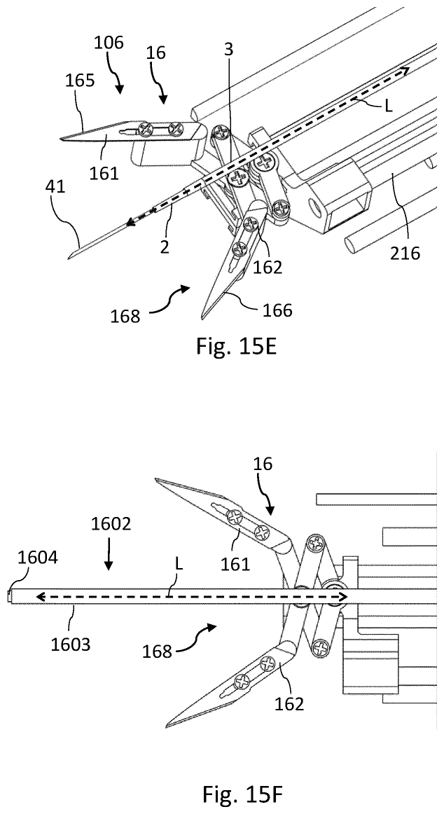

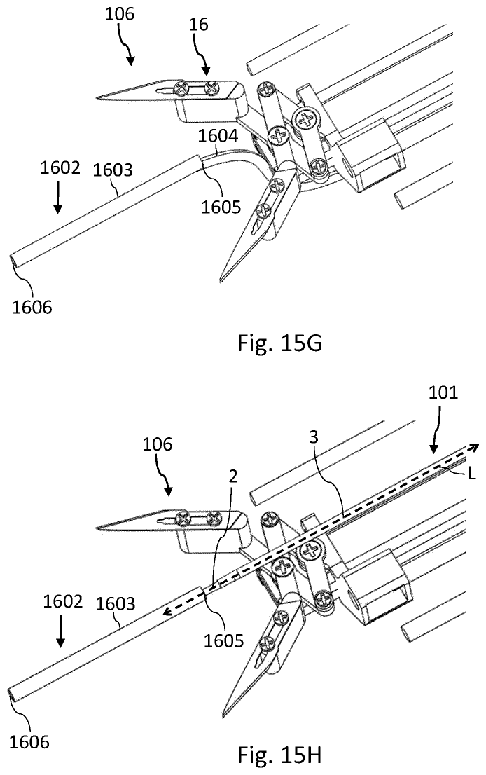

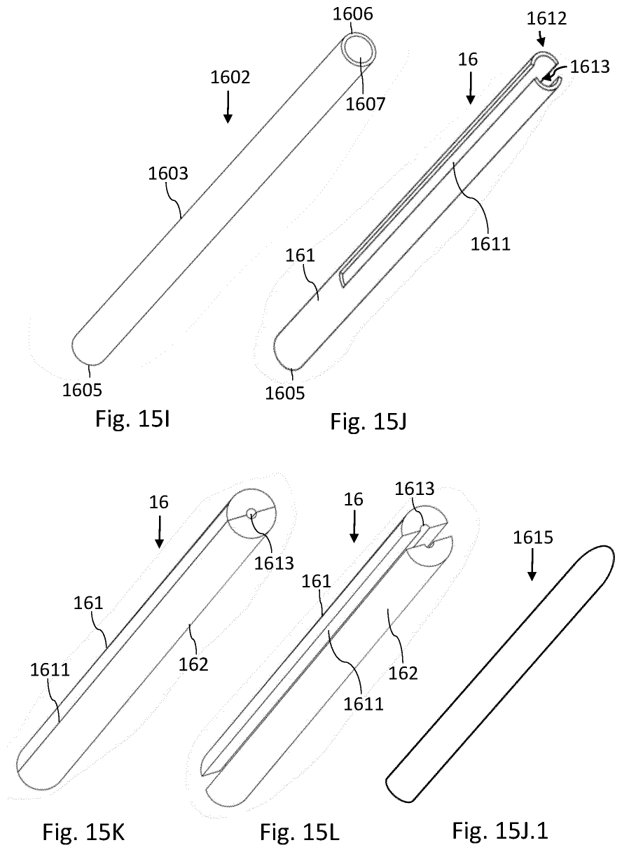

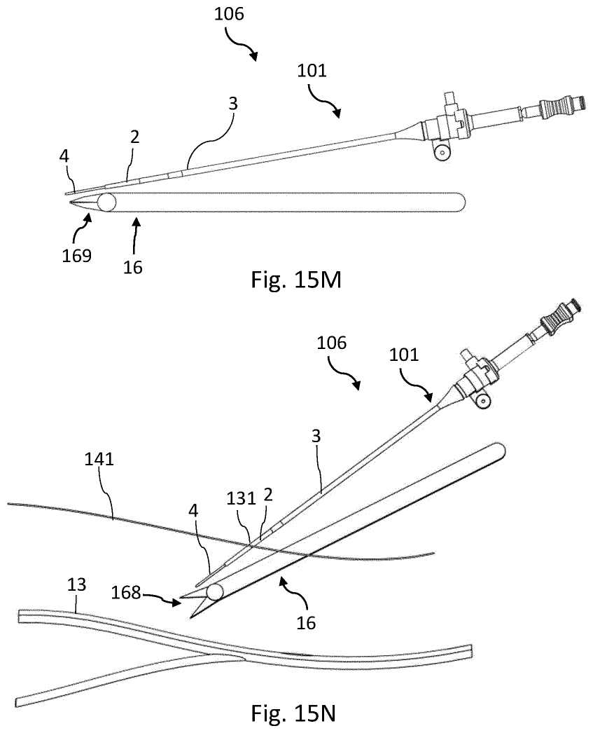

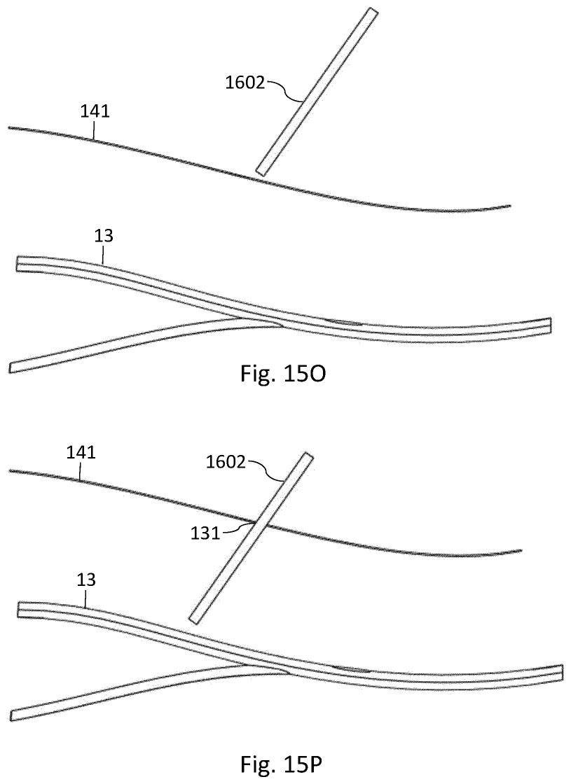

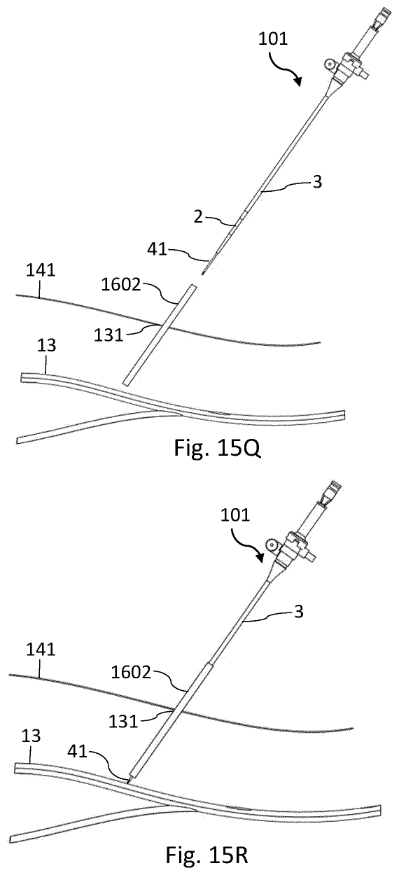

The FIGS. 15A and 15B show a top view of the system 104 according to FIGS. 14A to 14C and a method of advancement and retraction of the introducer assembly 1 via the actuation unit 9 of the system 104.

The actuation unit 9 is configured to move translationally in a proximal direction P and in a distal direction D of the system 104 along the centrally arranged guiding element 211 (see e.g. FIGS. 15A and 15B). In FIGS. 14A-14C the actuation unit 9 is configured for engaging and moving the third mounting section 183 connected to the guiding unit 4 by engaging the guiding elements 212, 214 via the rollers 92. Since the mounting sections 181, 182, 183 are sequentially movably arranged along the central guiding element 211, the actuation unit 9 is configured to move all three mounting sections 181, 182, 183 in unison along a distal direction D as shown in FIG. 15A. This allows the sheath 3, the dilator 2, and the guiding unit 4 to be moved in unison while being arranged at predetermined positions with respect to each other. The sheath 3, the dilator 2, and the guiding unit 4 in the predetermined positions are arranged such that a distal tip 44 of the guiding unit 4 extends from a distal opening 22 of the dilator 2 and a distal tip of the dilator 2 extends from a distal opening 32 of the sheath 3. The predetermined positions are a direct consequence of the design, in particular a longitudinal length, of the corresponding mounting sections 181, 182, 183.

As shown in FIG. 15A, by advancing the introducer assembly 1 in the predetermined positions via the actuation unit 9, the introducer assembly 1 may be inserted through a patient's skin into a body duct.

When the third mounting section 183 engages the second mounting section 182, a mechanical coupling 8 is formed by a hollow cylindrical element of the dilator 2 and a cylindrical element of the guiding unit 4 which engage with each other and couple the movement of the dilator 2 and the guiding unit 4. However, the mechanical coupling 8 may alternatively be arranged on the mounting sections 181, 182, 183 to the same effect.

FIG. 14B shows that the cutting element 16 can be arranged in a retracted state to avoid unintentional injury. The cutting element 16 is convertible to an extended state, shown in FIG. 14C, in which the cutting element 16 protrudes in a distal direction for performing an incision at a insertion site by the guiding unit 4. The cutting element 16 may either be extended automatically corresponding to the advancement of the guiding unit 4 or operated independently by the processing unit.

The cutting element 16 is configured to make the incision by placing its cutting edge at the insertion site of the guiding unit 4 and making the incision from there. The incision by the cutting element 16 is of a sufficient size to allow for atraumatic widening/insertion of the dilator 2 and subsequently the sheath 3.

FIG. 15B shows that once the sheath 3 was completely advanced, e.g. into the body duct, the dilator 2 and the guiding unit 4 can be retracted via the actuation unit 9 due to being coupled together with the mechanical coupling 8. However, the sheath 3 remains in place so that the surgical percutaneous procedure can be performed after removal of the system 104. This design of the system 104 allows for an atraumatic, preferably fully automated, catheterization using only a single actuation unit 9.

To disengage the biomedical device 101 from the system 104 after successful percutaneous catheterization, an engagement member 19 may be converted from an engagement configuration to a disengagement configuration by a processing unit (not shown in FIGS. 14A-15B). All three mounting sections 181, 182, 182 are equipped with a corresponding engagement member 19. The engagement member 19 in FIGS. 14A-15B is formed by a latch that can be extended to release the sheath 3, dilatator 2, and guiding unit 4 from the snap-fit mechanism of the mounting sections 181, 182, 183.

Subsequently, after detachment of the sheath 3, the dilator 2, and the guiding unit 4 via the engagement members 19, the system 104 can be removed, such that clinicians can carry out surgical interventions following catheterization.

The whole process of catheterization in FIGS. 14A-15B can be controlled via a processing unit which operates the actuation unit 9, the shearing unit, in particular the cutting element 16, and optionally the engagement members 19.

The FIGS. 15C-15H show a perspective side view of a system 106 for carrying out a percutaneous catheterization which has a shearing unit 16 at a distal tip of the system 106 for performing a sheared aperture. The shearing unit 16 has two shearing elements 161, 162 which are formed by two blades with an outwardly facing cutting edge 165, 166 respectively. The shearing elements 161, 162 are attached to a common fulcrum point 163 via bars which extend across the fulcrum point 163.

The FIGS. 15C-15H show that the shearing elements 161, 162 are arranged at an angle with respect to the bars in a range between 15°-60° for optimizing the pivoting movement of the shearing elements 161, 162. Thus, longitudinal axes of the shearing elements 161, 162 are further arranged at an angle with respect to each other in a closed configuration 169 (see FIG. 15C). This allows the first part of the pivoting movement of the shearing elements 161, 162 during conversion to an open configuration 168 (see FIG. 15D), to have a minor distal component as well as a radially outward component.

The ends of the bars are attached to two separate bars leading to a second fulcrum point 164 which is connected to an actuation section 167. The attachment of the shearing elements 161, 162 to the actuation section 167 forms a conversion mechanism which is configured to allow a pivoting movement of the shearing elements 161, 162 if the actuation section 167 is translationally advanced/retracted (see FIGS. 15C and 15D). This allows the shearing unit 16 to be converted between the closed configuration 169 and the open configuration 168.

In the closed configuration 169 in FIG. 15C, the distal end of the shearing elements 161, 162 converge to form a sharp distal tip 1601 which is adapted for puncturing an insertion site of the patient's skin.

In a different, embodiment the shearing elements 161, 162 may e.g. have a distal ends formed by T-shapes and are adapted for performing a blunt dissection by a rotational and/or translational movement of the shearing elements 161, 162.

The shearing unit 16 is mounted to a guiding element formed by a guide rail 216 of the system 106. The shearing unit 16 is actuatable by an actuation unit 9 (see e.g. FIGS. 14A-15B) by rotating a threaded surface of the guide rail 216 for retracting/advancing the shearing unit 16. This allows the shearing unit 16 to be advanced to perform a sheared aperture, in particular an incision at the insertion site, and retracted after performing the sheared aperture.

In FIGS. 15A-15H, the shearing unit 16 is advanceable along a longitudinal axis L of the system 106. The system 106 is configured such that the distal tip 1601 of the shearing unit 16 is arrangeable at an insertion site at a spatial position where the components of an introducer assembly such as a catheter, a dilator, a puncturing element, and an outer guiding unit may be inserted into the insertion site. Thus, the shearing unit 16 may facilitate the insertion of the introducer assembly during catheterization.

The FIG. 15E shows that a puncturing element 41, a dilator 2, and a sheath 3 may be advanceable by an actuation unit along the longitudinal axis L into an insertion site simultaneously to, prior, or after performing the sheared aperture via the shearing unit 16.

The FIG. 15F shows that an outer guiding unit 1602 may be advanced into the insertion site up to a proximal position close to a body duct to be catheterized (see FIGS. 15N-15Q). The outer guiding unit 1602 comprises an outer tubular member 1603 and an inner tubular member 1604 which are advanceable/retractable independently from each other via an actuation unit 9 (see e.g. FIGS. 14A-15B). The outer tubular member 1603 is formed by a stiffer material than the inner tubular member 1604 and has an inner lumen for receiving the inner tubular member 1604 and medical instruments, in particular the previously described biomedical device.

The inner tubular member 1604 is formed to seal the lumen of the outer tubular member 1603 during advancement of the outer guiding unit 1602 through the insertion site. Subsequently, the inner tubular member 1604 can be retracted via the actuation unit to clear the lumen while the outer tubular member 1603 remains within the body of the patient. This allows a reliable access to the vascular body duct even during catheterization or even after the catheterization was completed since medical/surgical instruments may be exchanged or reintroduced while the outer tubular member 1603 remains within the patient.

The FIGS. 15G and 15H show the system 106 deploying an embodiment of the outer guiding unit 1602 which has an outer tubular member 1603 which has a longitudinal length between 4 cm-8 cm and a lumen with a proximal opening 1605 and a distal opening 1606. A more flexible inner tubular member 1604 of the outer guiding unit 1602 is partially arranged within the lumen for sealing the lumen during insertion into the insertion site. A proximal section of the inner tubular member 1604 protrudes from the proximal end 1605 of the outer tubular member in a bent manner such that a longitudinal direction for insertion of components of the introducer assembly is not blocked by the inner tubular member 1604.

Subsequently to insertion of the guiding unit 1602, the inner tubular member 1604 can be retracted from the lumen at the proximal end 1605 as shown in FIG. 15H, e.g. via an actuation unit, along the bent spatial trajectory shown in FIG. 15G. This allows the components of the introducer assembly to be introduced through the lumen of the outer guiding unit 1602 through the insertion site into the patient for carrying out the catheterization. The FIG. 15H shows that a sheath 3, a dilator 2 of a biomedical device 101 can be introduced through the outer tubular member 1603 along a common longitudinal axis L of the biomedical device 101 and the outer tubular member 1603.

The FIG. 15I shows a perspective view of an embodiment of the guiding unit 1602 which is formed by an outer tubular member 1603 which has at least one sealing element, in particular two inner sealing elements, formed by an inflatable balloon or diaphragm made of silicon to allow for insertion of medical/surgical instruments and blocking outflow of blood of a patient.

The FIGS. 15J-15L show perspective side views of a first embodiments of a shearing unit 16 having a non-rotationally symmetric cross-section at its blunt distal tip 1612 and a second embodiment of a shearing unit 16 comprising two shearing elements 161, 162 which are radially movable with respect to each other for performing a sheared aperture

The FIG. 15J shows the first embodiment of the shearing unit 16 which is formed by a tubular shearing element 161 having two longitudinal slits 1611 which extend from the blunt distal tip 1612 of the tubular shearing element 161 in a longitudinal direction to an intermediate section of the tubular shearing element 161. The longitudinal slits 1611 are arranged on opposing sides of the shearing element 161. The blunt distal tip 1612 of the shearing element 161 has a non-rotationally symmetric oval cross-sectional shape such that a rotation via an actuation unit (not shown in FIG. 15J) creates shear forces to perform sheared apertures by blunt dissection at an insertion site.

A system using the shearing unit 16 of FIG. 15J may

further comprise a blunt inserter 1615 (see FIG. 15J.1) which may be inserted into an inner lumen 1613 of the shearing element 161 for enhancing the structural integrity during rotation of the shearing unit 16.

The actuation unit and a processing unit of a system for driving the rotation of the shearing unit 16 in FIG. 15J is preferably configured for translationally moving the shearing unit 16 in a distal direction and rotating the shearing unit 16 in a sequential or simultaneous manner. In a preferred embodiment, the rotation of the shearing unit 16 is carried out by sequentially rotating in a first angular direction around a longitudinal axis of the shearing unit 16 and subsequently rotating in a second angular direction opposing to the first angular direction. The rotational range of one sequential step preferably does not exceed more than a complete rotation of 360°, in particular not exceeding 180° in one angular direction, or even not exceeding 90° in one angular direction. A rapid switching of angular directions of the orientation further enhances the exerted shear forces and facilitates performing sheared apertures by blunt dissection.

The FIGS. 15K and 15L show a second embodiment of the shearing unit 16 which has two half cylindrical shearing elements 161, 162 which are separated from each other by longitudinal slits 1611 on opposing sides extending over the entire length of the shearing unit 16.

The FIG. 15K shows that the shearing elements 161, 162 enclose an inner lumen 1613 in a closed configuration of the shearing unit 16 and FIG. 15L shows that the shearing elements 161, 162 are movable radially outwardly with respect to each other to convert the shearing unit 16 to an open configuration. The inner lumen 1613 of the shearing unit 16 has a small cross-sectional dimension, e.g. less than 20% of a maximum cross-sectional dimension of the shearing unit 16, such that the inner lumen 1613 may be used as an entry point for components of a biomedical device or a blunt inserter 1615 (see FIG. 15J.1). This allows the shearing unit 16 in FIGS. 15K and 15L to be introduced through an insertion site in the closed configuration shown in FIG. 15K. Subsequently, the shearing unit 16 can be radially expanded, e.g. by insertion of the blunt inserter 1615 to convert the shearing unit 16 to the open configuration.

An actuation unit and processing unit of the system comprising the shearing unit 16 of FIGS. 15K and 15L is preferably configured to sequentially advance and convert the shearing unit 16 by advancing the shearing unit 16 in the closed configuration and then advance a blunt inserter 1615 or even a biomedical device in the inner lumen 1613 to convert the shearing unit 16 to the open configuration and perform a blunt dissection. Subsequently, the shearing unit 16 may be converted to the closed configuration again to be advanced deeper into the tissue by retracting the blunt inserter 1615 or biomedical device.

The processing unit may be configured to repeat this procedure in a fully automated manner a plurality of times to perform a plurality of blunt dissections by repeatedly advancing the shearing unit 16 deeper into the tissue until a catheterization of the body duct can be performed.

This expandable nature of the shearing unit 16 further enables insertion of medical/surgical instrument with different cross-sectional dimensions to be inserted through the insertion site to the body duct since the shearing elements 161, 162 expand accordingly.

The FIG. 15J.1 shows a blunt inserter 1615 with a rounded distal tip. A system for performing sheared apertures may comprise the blunt inserter 1615 of FIG. 15J.1 for enhancing the structural integrity of a rotatable shearing unit 16, in particular when rapidly changing an angular direction of rotation of a rotatable shearing unit (see FIG. 15J). Alternatively, the system may comprise the blunt inserter 1615 for converting the shearing unit 16 from a closed to an open configuration by repeatedly advancing or retracting the blunt inserter 1615 (see FIG. 15J.1).

The FIGS. 15M and 15N show a schematic side view of a system 106 for sequentially advancing a biomedical device 101 and a shearing unit 16 through an insertion site 131 the skin 141 of a patient up to a body duct 13 using a shearing unit 16 for repeatedly performing sheared apertures.

The system 106 has an actuation unit and a processing unit (not shown in FIGS. 15M and 15N) which are adapted for operating the translational movement of the biomedical device 101 and the shearing unit 16. The biomedical device 101 has an outer sheath 3, a dilator 2, and an inner guiding unit 4 which are nested within each other. The actuation unit and the processing unit are further adapted to convert the shearing unit 16 between a closed configuration 169 (see FIG. 15M) and an open configuration 168 (see FIG. 15N). The introduction of the biomedical device 101 is carried out by the processing unit and actuation unit being adapted to sequentially advance the shearing unit 16 in the closed configuration 169 and subsequently converting the shearing unit 16 to the open configuration 168. The shearing unit 16 has at least two shearing elements 161, 162 which converge to form at least one distal tip in the closed configuration 169 such that it can be advanced through the tissue sequentially along a longitudinal direction of the shearing unit 16.

The shearing elements 161, 162 of the shearing unit 16 in FIGS. 15M and 15N further have two outwardly facing blunt edges, such that the shearing unit 16 is adapted for forming a sheared aperture of the tissue by carrying out a blunt dissection when converted from the closed configuration 169 to the open configuration 168. The processing unit and actuation unit are further adapted to sequentially advance the biomedical device 101 once a further distal sheared aperture was performed. This procedure can be repeated, in particular in a fully automated manner controlled by the processing unit, until the biomedical device 101 was introduced into the body duct 13, in particular into a femoral artery of the patient.

This allows for a more atraumatic vascular catheterization procedure to introduce the biomedical device 101.

The FIGS. 15M and 15N show the shearing unit 16 arranged at an angle with respect to a longitudinal direction of the biomedical device 101 for better visibility. However, in a preferred embodiment, the system 106 may be adapted for advancing the shearing unit 16 and the biomedical device 101 along a common longitudinal direction.

The system 106 of FIGS. 15M and 15N may alternatively or additionally used for advancement and intracorporeal positioning of an outer guiding unit 1602 (see FIGS. 15O-15R).

The FIGS. 15O-15R show a schematic side view of inserting and placing an outer guiding unit 1602 through an insertion site 131 almost up to a body duct 13 formed by the femoral artery and subsequent catheterization using a biomedical device 101. The outer guiding unit 1602 may be positioned through the skin 141 of the patient at an insertion site 131 as previously described in FIGS. 15M and 15N with regard to a biomedical device 101.

The outer guiding unit 1602 may be formed according to one of the three embodiments in FIGS. 15I-15L to inhibit blood efflux while simultaneously facilitating the insertion of medical/surgical instruments, such as the biomedical device 101. This enables the outer guiding unit simplifies the introduction and exchange of medical/surgical instruments during surgical procedures.

The FIGS. 15O and 15R show that the biomedical device 101 can be advanced through the outer guiding unit 1602. This process is preferably carried out by a previously described system, e.g. via a processing unit controlling an actuation unit of the system carrying out the insertion of the outer guiding unit 1602 and biomedical device 101 in a fully automated manner.

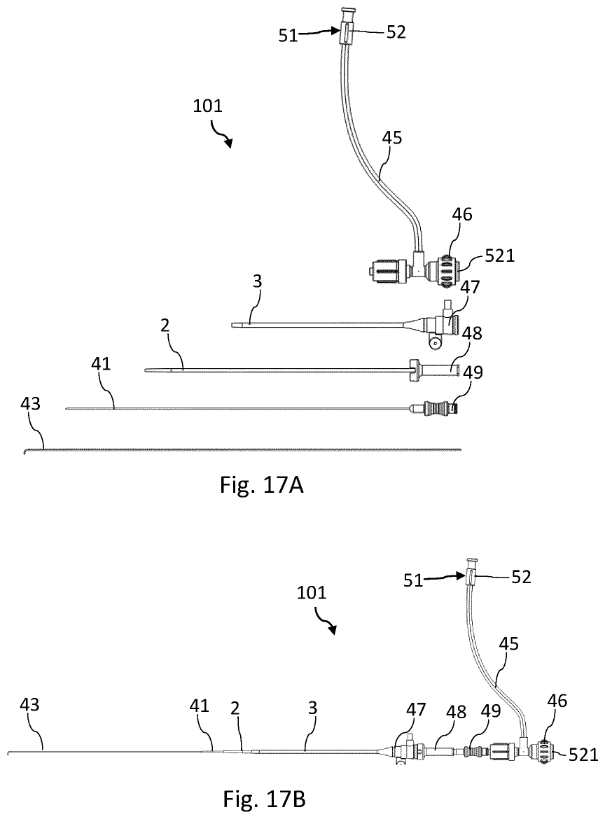

The biomedical device 101 in FIGS. 15O and 15R is described in further detail in FIGS. 17A and 17B.

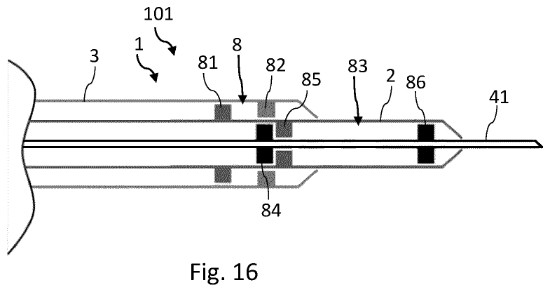

FIG. 16 shows a longitudinal cross-sectional view of an embodiment of the biomedical device 101 with an introducer assembly 1 comprising a sheath 3, a dilator 2, a puncturing element 41, and two mechanical couplings 8, 83. The first coupling 8 is formed by a first and second annular coupling element 81, 82 between the sheath 3 and the dilator 2 of the introducer assembly 1. The second coupling 83 is formed by three coupling elements 84, 85, 86 arranged on the dilator and the puncturing element 41. The coupling elements 81, 82, 84, 85, 86 are essentially formed as the previously described coupling elements in FIG. 7.

The first and second coupling elements 84, 85 of the second coupling 83 are arranged such that they engage each other at a specific position when the puncturing element 41 is moved along a distal direction. This allows to limit the length of the puncturing element 41 extending from a distal end of the dilator and at the same time allows for moving the puncturing element 41 and the dilator 2 in unison once the first and second coupling elements 84, 85 engage each other. In addition, the first and second coupling elements 81, 82 of the first coupling 8, once engaging each other, allow for movement of the dilator 2 and the sheath 3 in unison. Consequently, the puncturing element 41, the dilator 2, and the sheath 3 may be advanced simultaneously by only actively actuating the puncturing element 41, e.g. via an actuation unit.

Moreover, when retracting the puncturing element 41, the second and third coupling elements 85, 86 of the puncturing element 41 and dilator 2 engage each other, such that the puncturing element 41 and the dilator 2 may be retracted in unison. However, at the same time, no coupling element for coupling of the movement of the dilator 2 and the sheath 3 is provided, such that the dilator 2 and puncturing element 41 may be retracted and removed with the sheath 3 remaining stationary, e.g. within a body duct of a patient.

The FIGS. 17A and 17B show a top view of an embodiment of the biomedical device 101 which comprises an introducer assembly 1. The introducer assembly 1 has a guidewire 43, a puncturing element 41 formed by a cannula, a dilator 2, a sheath 3, and a side port 45 separate from each other and nested within each other respectively. The puncturing element 41 may have a sufficient longitudinal elongation such that the dilator may be directly advanced into a body duct for catheterization, preferably a longitudinal elongation of at least 6 cm, 8 cm, or 12 cm. The puncturing element 41, the dilator 2, the sheath 3, and/or the side port may have a proximal hub 46, 47, 48, 49 respectively. The embodiment of FIG. 17B shows that the guidewire 43 can be nested within the puncturing element 41, the puncturing element 41 can be nested within the dilator 2, and the dilator 2 can be nested within the sheath 3. FIG. 17B shows that the proximal hub 46 of the side port 45 may be attached proximal to the hub 49 of the puncturing element 41. The hub 49 of the puncturing element 41 may be attached proximal to the hub 48 of the dilator 2. The hub 48 of the dilator 2 may be attached proximal to the hub 47 of the sheath 3. If the proximal hubs 48, 49 of the dilator 2 and the puncturing element 41 are connected, the puncturing element 41 may protrude from a distal opening of the dilator which may allow the puncturing element 41 and dilator to be advanced in a longitudinal direction in unison.

A cavity enclosed by the side port 45 may be connectable in a fluid communicating manner with a lumen of the puncturing element 41. A hemostasis valve 52 may be arranged within the cavity of the side port 45. The FIGS. 17A and 17B show that the hemostasis valve 52 may be arranged at an outward end of a lateral branch of the side port 45. The hemostasis valve 52 arranged laterally in the side port 45 may allow for administering drugs in parallel to movement of the guidewire 43. The proximal hub 46 of the side port 45 may have a second hemostasis valve 521 which allows the guidewire 43 to be advanced/retracted while maintaining the pressure within the cavity of the side port 45 and the lumen of the puncturing element 41. In addition, a pressure sensor 51 for detecting the intraluminal pressure may be arranged within the hemostasis valves 52, 521. In the FIGS. 17A and 17B, the pressure sensor 51 is arranged in the laterally arranged hemostasis valve 52.

The proximal hub(s) 46, 47, 48, 49 may be formed with a shape corresponding to a shape of a mounting section, e.g. corresponding to an open shape of the mounting section, of a sterile part of a system for percutaneous catheterization comprising the biomedical device 101. At least two proximal hubs may comprise a coupling, the coupling in particular comprising a plurality of coupling elements, such that the movement of the hubs 46, 47, 48, 49 may be coupled with respect to each other.

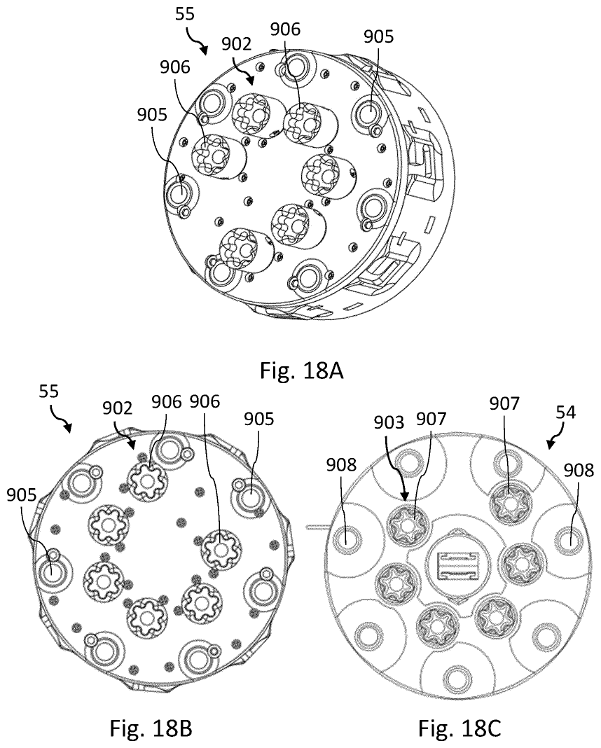

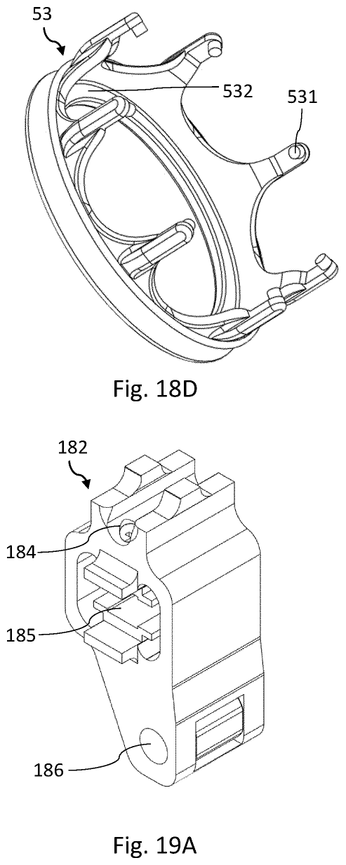

The FIGS. 18A and 18B show a perspective view and a top view of an embodiment of a non-sterile part 55 of a system for percutaneous catheterization comprising a first transmission unit 902 of an actuation unit. The first transmission unit 902 may be adapted to form a plurality of connections for transmission of a rotational movement when being connected via at least one engagement structure 906, e.g. via six engagement structures 906 of the first transmission 902 as shown in FIGS. 18A and 18B, to a second transmission unit 903 (see FIG. 18C). The non-sterile part 55 may have a plurality of recessed, protruding and/or magnetic connecting elements, e.g. six magnetic connecting elements 905 as shown in FIGS. 18A and 18B. The engagement structures 906 of the non-sterile part 55 may be formed for releasably engaging engagement structures 907 of the sterile part 54 (see FIG. 18C) for transmission of the rotational movement, e.g. for actuation of a component of the introducer assembly/mounting sections of the system.

The non-sterile part 55 may comprise a plurality of actuators of the actuation unit for driving the rotational movement. The non-sterile part 55 may comprise a processing unit for controlling the actuation unit, in particular in a fully automated manner (not shown in the FIGS. 18A and 18B).

The FIG. 18C shows a top view of an embodiment of a sterile part 54 comprising a second transmission unit 903 of the actuation unit. The second transmission unit 903 may have a plurality of engagement structures 907 which are correspondingly formed and positioned to engage the engagement structures 906 of the non-sterile part 55 (see FIGS. 18A and 18B) for transmission of a rotational movement. FIG. 18C shows that the sterile part 54 may comprise connecting elements 908 which are correspondingly formed and positioned to the connecting elements 905 of the non-sterile part 55. In FIG. 18C the connecting elements 908 of the sterile part 54 are magnetic such that the sterile and non-sterile part 54, 55 may be securely connectable with respect to each other. The magnetic connecting elements 905, 908 may be embedded in the surfaces of the sterile and non-sterile part 54, 55.

The engagement structures 906, 907 and/or the connecting elements 905, 908 of the sterile and non-sterile part 55 may be arranged asymmetrically such that the sterile and non-sterile part 54, 55 are only connectable at a specific orientation with respect to each other. This ensures that the actuation unit may connect specific actuators to actuate specific parts of the introducer unit/mounting sections of the system.

The FIG. 18D shows a perspective view of an embodiment of a retaining element 53 adapted for retaining the sterile part 54 of FIG. 18C and the non-sterile part 55 of FIGS. 18A and 18B in a connected state together. The retaining element 53 may have an annular shape as shown in FIG. 18D. The retaining element 53 may have a contacting surface 532 for engaging the sterile part 54. The retaining element 53 may have a plurality of connecting elements. FIG. 18D shows that the connecting elements may be formed by bayonet connecting elements 531 which may be arranged at different circumferential positions of the retaining element 53. This may allow the retaining element 53 to be rotated with respect to the sterile and/or non-sterile part 54, 55 to be connected to the sterile or non-sterile part 54, 55 by locking all bayonet connecting elements 531 simultaneously.

FIG. 18D shows that the circumferential positions of the bayonet connecting elements may be arranged equidistantly spaced apart from each other along the annular shape of the retaining element 53. The bayonet connecting elements 531 may be male bayonet connecting elements 531 as shown in FIG. 18D which are adapted to be connected with female bayonet connecting elements of the sterile and/or non-sterile part 54, 55.

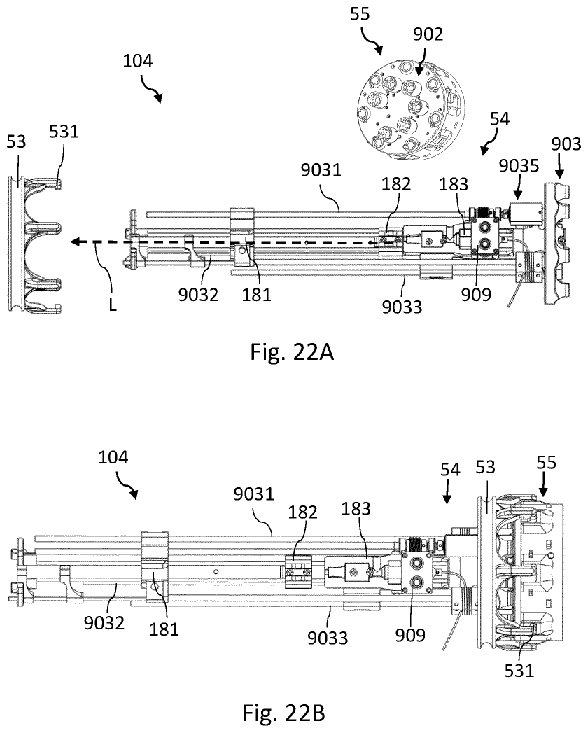

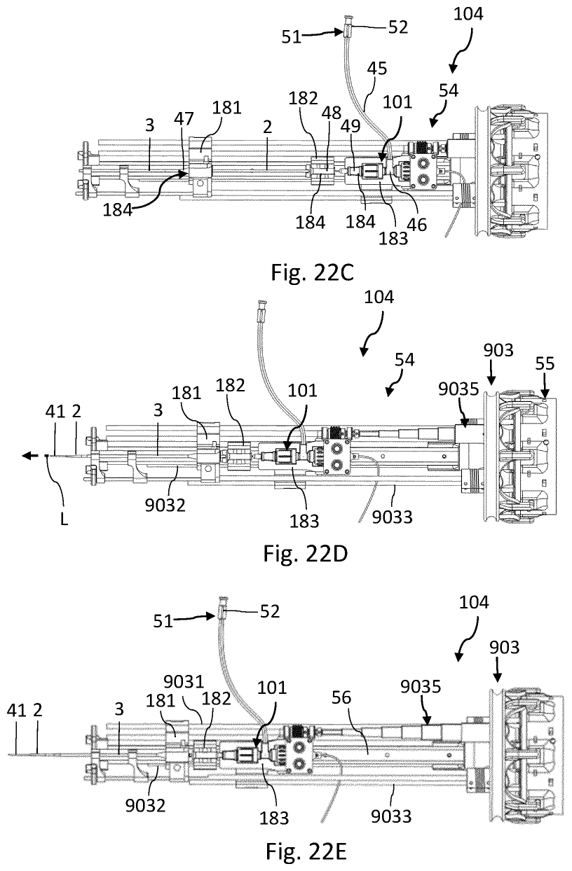

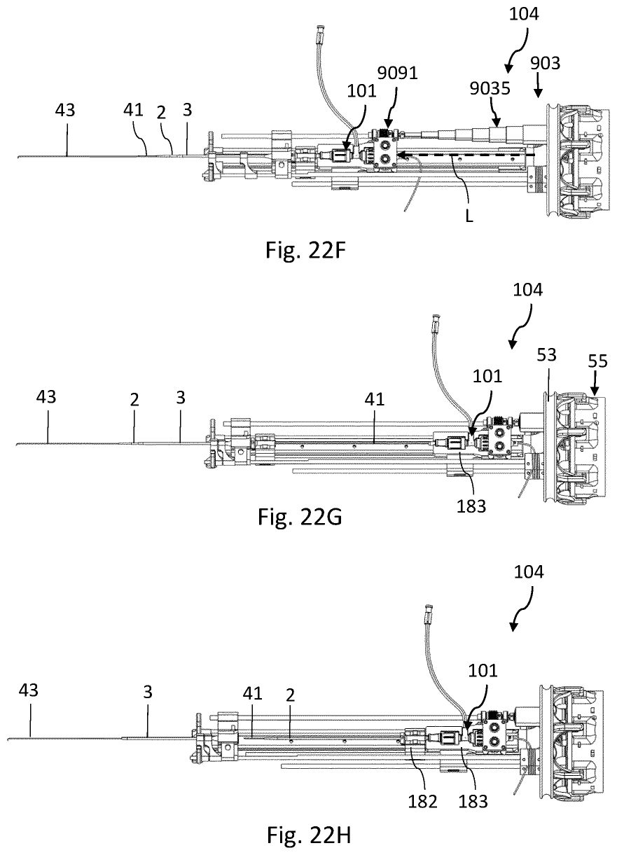

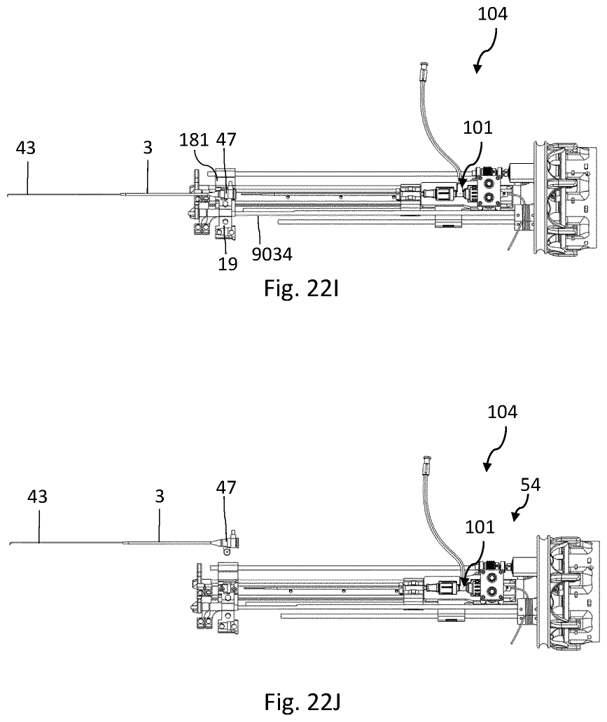

The retaining element 53 may be positioned with the contacting surface 532 contacting the sterile part 54 while engaging a plurality of female bayonet connecting elements on the non-sterile part 55 (see FIGS. 22B and 22C). This allows a secure and simple retaining arrangement of the sterile and non-sterile part 54, 55.

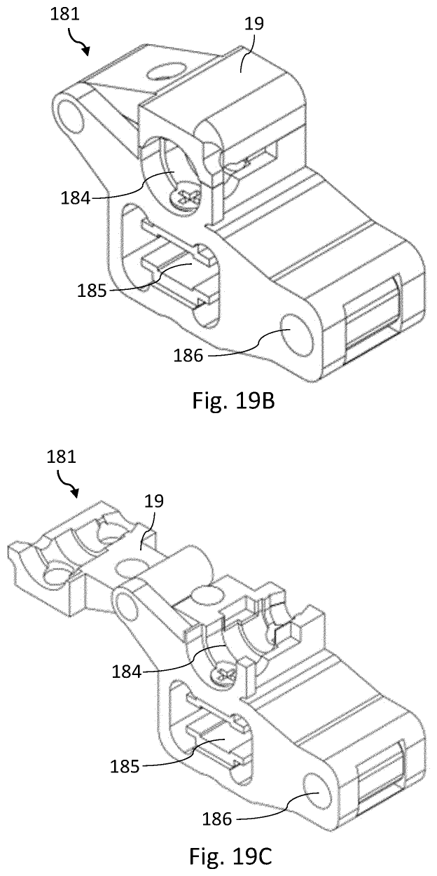

The FIG. 19A shows a perspective side view of an embodiment of a mounting section 182 for mounting the dilator 2 (see e.g. FIGS. 17A and 17B) of a system for percutaneous catheterization. The mounting section 182 may have a partially open shape 184 in a radial direction to receive a proximal hub 48 of the dilator 2 for actuation of the dilator 2 in a longitudinal direction of the introducer assembly.

The mounting section 182 may further comprise a guiding region 185. The guiding region 185 may be formed by a through passage in the longitudinal direction as shown in FIG. 19A which may be connectable to receive a guiding structure, e.g. a guide rail 56 (see FIGS. 22A-22J). The guiding region 185 may be adapted to guide the mounting section 182 parallel to the longitudinal direction. The guiding region 185 may be formed to receive a non-circular guiding structure, e.g. having a rectangular cross-section, in a specific spatial orientation such that the mounting section 182 is not rotatable in an orthogonal plane with respect to the longitudinal direction when being moved along the guiding structure.

The mounting section 182 may further have a transmission region 186 which may be adapted to convert a rotational actuation, e.g. via a torque shaft 9031, 9032, 9033 of the second transmission unit (see FIGS. 22A-22J), into a movement of the mounting section 182 in the longitudinal direction. The FIG. 19A shows that the transmission region 186 may comprise a threaded passage parallel to the longitudinal direction which may e.g. be drivable by a threaded spindle shaft of the second transmission unit for the longitudinal actuation of the mounting section 182.

The FIGS. 19B and 19C show a perspective side view of an embodiment of a mounting section 181 of the sheath of a system for percutaneous catheterization. The mounting section 181 may have an engagement member 19 which is convertible between an engagement configuration (see FIG. 19B) and a disengagement configuration (see FIG. 19C). In the engagement configuration, the engagement member 19 may enclose a radially partially open shape 184 of the mounting section 181 such that a sheath, in particular a proximal hub of the sheath, may be enclosed. In the disengagement configuration of the engagement member 19 shown in FIG. 19B, the sheath may be radially released from the mounting section 181, e.g. after completing the percutaneous catheterization. The engagement member 19 may be convertible by the second transmission unit, e.g. by a torque shaft 9034 of the second transmission unit 903 (see FIG. 22I), for converting the engagement member 19 by rotation driven by the actuation unit.

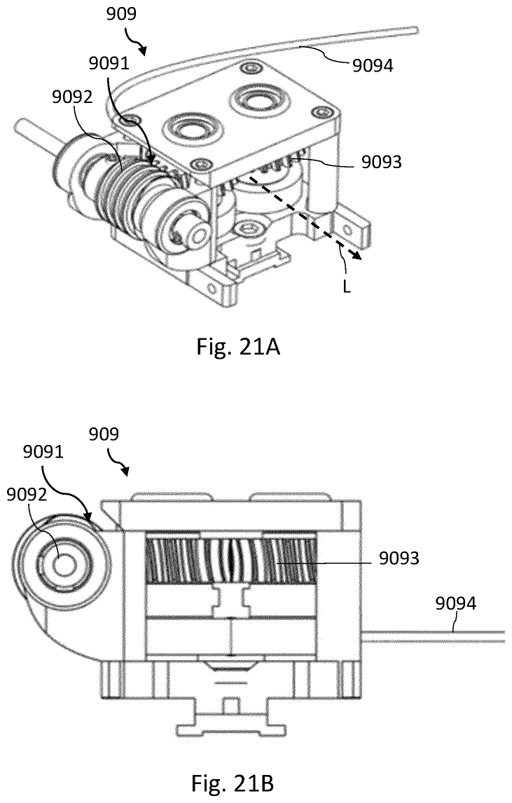

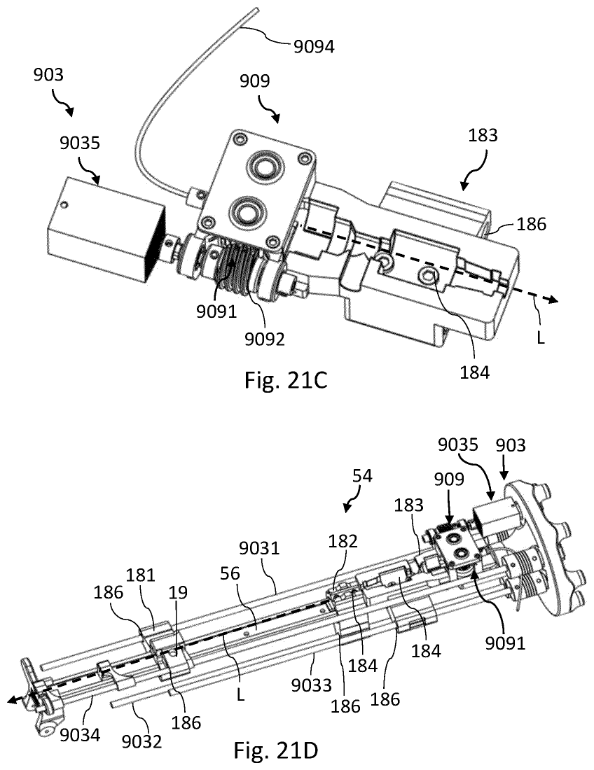

FIGS. 19B and 19C show that a guiding region 185 of the mounting section 181 may be connectable to the guiding structure as previously described in FIG. 19A, preferably a common guiding structure, e.g. guide rail 56 (see FIG. 21D), which also guides the mounting section 182 of the dilator.

A transmission region 186 of the mounting section 181 may also be adapted to convert a rotational actuation, e.g. via the second transmission unit, into a movement of the mounting section 181 in the longitudinal direction. However, the mounting section 181 as shown in FIGS. 19B and 19C may be formed such that the transmission region 186 is arranged at a different angular orientation/spatial position with respect to the guiding region 185 than the mounting section 182 of the dilator in FIG. 19A. This may allow for independent actuation of the mounting sections 181, 182. The transmission region 186 in FIGS. 19B and 19C which may comprise a threaded through passage parallel to the longitudinal direction. The threaded passage may e.g. be adapted to receive a different spindle shaft 9032 of the second transmission unit 903 for the longitudinal actuation than the mounting section 182 of the dilator (see FIG. 21D). This may allow the mounting sections 181, 182 to be actuated in the longitudinal direction via the actuation unit comprising the second transmission unit by rotation of different spindle shafts 9031, 9032 independently from each other.

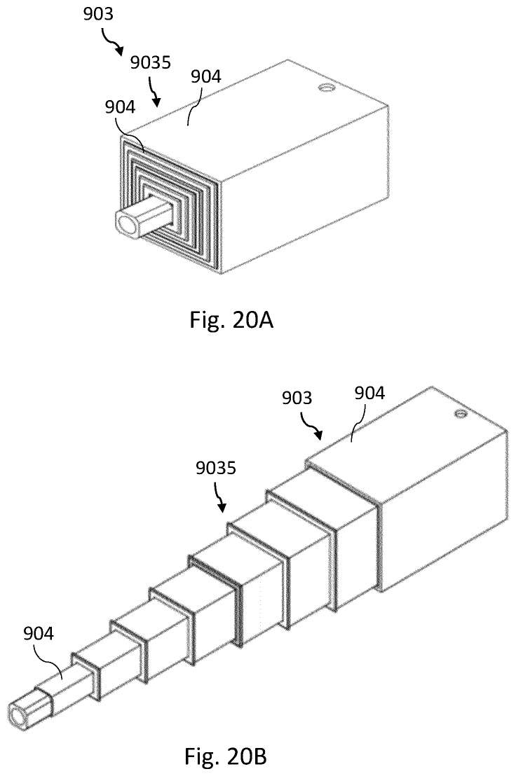

FIGS. 20A and 20B show a perspective side view of an embodiment of an extendable part 9035 of a second transmission unit 903 of an actuation unit of a system for percutaneous catheterization which is extendible in a longitudinal direction. The extendable part 9035 of the second transmission unit 903 may be formed by a plurality of transmission elements 904 which are nestable within each other. This allows that the second transmission unit 903 may be convertible between a retracted state and an extended state shown in FIGS. 20A and 20B respectively. A distal end of the extendable part 9035 of the second transmission unit 903 may be connected to a mounting section 183 of the guiding unit, in particular of the puncturing element (see FIGS. 21A and 21B). The extendable part 9035 of the second transmission unit 903 may be adapted to be converted between the extended and retracted state by moving the mounting section 183 of the guiding unit, e.g. the guidewire, in a longitudinal direction of an introducer assembly. A proximal side of the second transmission unit (not shown in FIGS. 20A and 20B) may have an engagement structure 907 (see FIG. 18C) for engaging an engagement structure 906 of a first transmission unit 902 of a non-sterile part 55 (see FIGS. 18A and 18B). This may allow an actuation unit comprising the first and second transmission unit 902, 903 (see FIG. 21D) to rotationally actuate the extendable part 9035 of the second transmission unit. For this reason, the transmission elements 904 may have an interlocking non-circular cross-sectional shape, e.g. a rectangular shape as shown in FIG. 20B. A rotation of a proximal most transmission element 904 may thus result in a rotation of a distal most transmission element 904 irrespective of the state of extension.

FIGS. 21A-21C show a perspective side view and a side view of an embodiment of a third transmission unit 909 of an actuation unit of a system for percutaneous catheterization and a perspective side view of a third transmission unit 909 of FIGS. 21A and 21B attached to a mounting section 183 for a guiding unit.

The third transmission unit 909 may have a gear system 9091 which is adapted to be connected to an extendable part 9035 of the second transmission unit 903 (see FIGS. 20A and 20B). The gear system 9091 may be adapted to convert a rotational actuation of the second transmission unit 903 which can be driven via an actuator connected to a first transmission unit 902 (see FIGS. 18A and 18B) for a longitudinal actuation of the guiding unit of the system, in particular of a guidewire 43 of the guiding unit (see FIG. 22F). The FIGS. 21A and 21B show that the gear system 9091 may comprise of a rotatably mounted threaded spindle 9092 which is adapted to drive two gear wheels 9093. The gear wheels 9093 may engage with each other in a drivable manner, in particular by means of splines. The interlocking gear wheels 9093 may be adapted to drive the guidewire of the guiding unit in a longitudinal direction L. The third transmission unit 909 may consequently convert a rotational movement of the second transmission unit 903 into a longitudinal movement of the guidewire. The FIGS. 21A and 21B show that the third transmission unit 909 may further comprise a guide 9094, e.g. a resilient tube, for guiding the guidewire which prevents buckling or kinking of the guidewire when being advanced/retracted via the third transmission unit 909.

The FIG. 21C shows that the mounting section 183 may have an open shape 184 for receiving the guiding unit, in particular the puncturing element. The open shape 184 may be formed to receive a proximal hub 49 of the puncturing element (see FIGS. 17A and 17B). In addition, the open shape 184 may be adapted to further receive a side port 45, in particular a proximal hub 46 of the side port 45 as shown in FIG. 21C. The FIG. 21C further shows that the threaded spindle shaft 9092 of the gear system 9091 may be connected to a distal end of extendable part 9035 of the second transmission unit 903 to actuate the guidewire in the longitudinal direction through the guide 9094. In addition, the third transmission unit 909 may be moved in unison with the mounting section 183 while the extendable part 9035 of the second transmission unit 903 is extended accordingly. The mounting section 183 may have a transmission region 186 and a guiding region (not shown in FIGS. 21A-21C). The guiding region may be formed similar to the previously described guiding regions 185 and adapted to guide the mounting section 183 along the longitudinal direction, e.g. along a guide rail (see FIGS. 19A-19C). The transmission region 186 may comprise a threaded passage parallel to the longitudinal direction L. The transmission region 186 may be adapted to receive a spindle shaft 9093 of the second transmission unit 903 for moving the mounting section 183 in the longitudinal direction L. In a preferred embodiment, the mounting sections 181, 182, 182 (see FIGS. 19A-19C) are independently movable in the longitudinal direction L by actuation of separate spindle shafts 9031, 9032, 9033 of the second transmission unit 903.

The FIG. 21D shows a perspective side view of an embodiment of a sterile part 54 of a system 104 for carrying out a percutaneous catheterization in an at least partially automated manner. The sterile part 54 may have three mounting sections 181, 182, 183. The first mounting section 181 may be adapted for mounting the sheath in an open shape and has an engagement member 19 which may be adaptable to be convertible between an engagement configuration for engaging and locking the sheath and a disengagement configuration for radially releasing the sheath (see FIGS. 19B and 19C). The engagement member 19 may be convertible by rotational actuation of a torque shaft 9034 of the second transmission unit 903 which is connectable to an actuator of the actuation unit. The actuator may be arranged within the non-sterile part 55 (see FIGS. 22A-22J). The second mounting section 182 may be adapted for mounting the dilator in an open shape 184 (see FIG. 19A). The third mounting section 183 may be adapted to mount the guiding unit, in particular the puncturing element formed by a cannula, and preferably the side port in an open shape 184 (see FIG. 21C).

The mounting sections 181, 182, 183 may be guided along a guiding structure such as a guide rail 56 shown in FIG. 21D. The FIG. 21D shows that all mounting sections 181, 182, 183 are arranged in series and guided along a common guide rail 56 of the sterile part 54 when moving in a longitudinal direction L of the sterile part 54. The mounting sections 181, 182, 183 may comprise guiding regions for receiving the guiding structure. The FIG. 21D shows that the guiding regions may comprise through holes which are formed correspondingly to receive the guide rail 56 such that the mounting sections 181, 182, 183 are non-rotatably movable along the guide rail 56 in the longitudinal direction L.

The FIG. 21D shows that the mounting sections 181, 182, 183 may all be connected to a separate torque shaft 9031, 9032, 9033, e.g. screw shafts, of the second transmission unit 903 at their transmission regions 186 respectively. The transmission regions 186 may have a threaded through hole parallel to the longitudinal direction for receiving the torque shafts 9031, 9032, 9033 as shown in FIG. 21D. The torque shafts 9031, 9032, 9033 may be rotatable independently from each other. This allows that the three mounting sections 181, 182, 183 may be actuatable independently from each other, e.g. by separate actuators of the actuation unit driving the second transmission unit. In a preferred embodiment, the actuators of the actuation unit are arranged in the non-sterile part (not shown in FIG. 21D).

The FIG. 21D shows that the sterile part 54 may have a third transmission unit 909 (see FIGS. 21A-21C). The third transmission unit 909 may be connected or connectable to the third mounting section 183 such that the third transmission unit 909 and the third mounting section 183 for mounting the guiding unit, e.g. the puncturing unit, may be movable in the longitudinal direction in unison together. The third transmission unit 909 may further be connected to an extendable part 9035 of the second transmission unit 903. In FIG. 21D, the extendable part 9035 is formed by nested elements which a rectangular cross-section. The extendable part 9035 may be adapted for actuating the third transmission unit 909 by rotating similar to a torque shaft but having a plurality of nested transmission elements 904 which interlock with each other when rotating (see FIGS. 20A and 20B). This allows a gear system 9091 of the third transmission unit 909 to convert a rotational movement via the extendable part 9035 to drive the movement of a guidewire in the longitudinal direction L.

The sheath, the dilator, and/or the guiding unit may have a proximal hub 47, 48, 49 which can be mounted in the mounting sections 181, 182, 183 (see FIGS. 17A and 17B). The open shapes 184 of the mounting sections 181, 182, 183 may be formed correspondingly to the proximal hub 47, 48, 49 of the sheath, dilator, and guiding unit. The open shape of the third mounting section 183 may further be formed to receive the proximal hub 47 of the side port 45.

The FIGS. 22A-22J show a top view of an embodiment of a system 104 comprising the sterile part 54 of FIG. 21D and a non-sterile part 55 carrying out the steps of percutaneous catheterization with a biomedical device 101. The first, the second, and the third mounting sections 181, 182, 183 may be movably attached to torque shafts 9031, 9032, 9033 at their transmission regions such that they are movable in the longitudinal direction L by an actuation unit comprising a first and second transmission unit 902, 903 independently from each other. The first transmission unit 902 of the non-sterile part 55 may have a plurality of engagement structures 906 which are adapted to be engaged to engagement structures 907 of the second transmission unit 903 of the sterile part 54 for transmitting an actuation movement, e.g. a rotational actuation movement (see FIGS. 18A-18C). In addition, a plurality of connecting elements 905 908 of the sterile and non-sterile part 54, 55 may be connectable. A third transmission unit 909 may be arranged attached to an extendable part 9035 of the second transmission unit 903 as previously described in FIG. 21D.

The FIGS. 22A and 22B show that the sterile and non-sterile part 54, 55 may be retained in a connected state by being connected via an annular retaining element 53 which may have a plurality of bayonet connecting elements 531. Actuators for driving the actuation unit may be arranged within the non-sterile part 55.

The FIG. 22C shows that the biomedical device 101 as described in FIGS. 17A and 17B may be mounted in the mounting sections 181, 182, 183. The biomedical device 101 may be comprised by the sterile part 54. The biomedical device 101 has a sheath 3, a dilator 2, and a guiding unit formed by a puncturing element 41. The biomedical device 101 may further comprise a side port 45 which may have a hemostasis valve 54 in the branched part of the side port 45 as shown in FIG. 22C. A pressure sensor 51 may be arranged within the hemostasis valve 52 for detecting an intraluminal pressure within a body duct by being fluid connected to a lumen of the puncturing element 41. The sheath 3, the dilator 3, the puncturing element 41 and the side port 45 may have a proximal hub 47, 48, 49, 46 which may be mounted within an open shape 184 of the respective mounting sections 181, 182, 183.

The FIG. 22D shows that the first transmission unit of the actuation unit arranged in the non-sterile part 55 may be actuatable to move the first and second mounting section 182, 183 in a distally in the longitudinal direction L. The first transmission unit may transmit a rotational motion via the engagement sections to the transmission unit 903 to control a rotational movement of the torque shafts 9032, 9033 of the second transmission unit 903. This allows to advance the first and second mounting sections 182, 183 such that the puncturing element 41 and the dilator 2 protrude from a distal tip. The puncturing element 41 may protrude a predetermined distance from the dilator 2 as shown in FIG. 22D. The extendable part 9035 of the second transmission unit 903 connected to the third mounting section 183 may be extended parallel to the longitudinal direction L by the distance the third mounting section 183 was moved as shown in FIG. 22D.

The FIG. 22E shows that the actuation unit may be actuated to control the torque shafts 9031, 9032, 9033 to advance the puncturing element 41, the dilator 2, and the sheath 3 to be advanced into a body duct (not shown in FIG. 22E). Prior or after the introduction of the puncturing element an incision may be made extending from or across the puncture site/future puncture site. This incision may be carried out by a clinician or via a shearing unit controlled by a processing unit. The shearing unit may be a cutting element 16 as previously described in FIGS. 14A-15B. The system, in particular the non-sterile part 54 in FIG. 21D, may comprise the cutting element 16 as previously described.

Since the mounting sections 181, 182, 183 may be arranged on a common guide rail 56, the movement may be coupled such that e.g. actuation of the third mounting section 183 moves the other mounting sections 181, 182 in the distal direction L. The introduction into the body duct may be guided via imaging means, in particular by an ultrasound probe.

The FIG. 22F shows that a guidewire 43 may be introduced through the lumen of the puncturing element 41 once the puncturing element 41 was introduced into the body duct. The guidewire 43 may be advanced in the distal direction L by rotating the extendable part 9035 of the second transmission unit 903. The rotation of the extendable part 9035 may drive a gear system 9091 to convert which converts the rotational movement to a translation movement of the guidewire 43. The gear system 9091 may comprise a rotatably mounted threaded spindle 9092 which may be coupled to the extendable part 9035 to drive gear wheels 9093 to actuate the guidewire 43 (see 21A-21C). This allows to move the guidewire 43 independently from the position of the third mounting section 183 without translationally moving the third transmission unit 909/third mounting section 183.

The FIG. 22G shows that the puncturing element 41 may be retracted by retracting the third mounting section 183 such that only the guidewire 43, the dilator 2, and the sheath 3 remains within the body duct.

The FIG. 22H shows that the dilator 2 may be retracted by retracting the second mounting section 182 such that only the sheath 3 and the guidewire 43 remain within the body duct.

The FIG. 22I shows that a fourth torque shaft 9034 of the second transmission unit 903 may be rotated for converting an engagement member of the first mounting section 181 from an engagement configuration retaining the sheath 3 to a release configuration for radially releasing the sheath 3 and a proximal hub 47 of the sheath 3 (see FIG. 17A and 17B and FIGS. 19A and 19B). The guidewire 43 may remain within a lumen of the sheath 3.

The FIG. 22J shows that the guidewire 43, the sheath 3, and the hub 47 may be radially released from the sterile part 54. This allows to complete the catheterization with the guidewire 43 and the sheath 3 within the body duct detached from the biomedical device 101 and allows a clinician to carry out a subsequent surgical procedure.

The system 104 in FIGS. 22A-22J may comprise a processing unit for controlling the actuation unit 9. The processing unit may further be adapted to drive the third transmission unit 909 via the second transmission unit 903 independently to carry out the above-described steps, in particular in a partially or fully automated manner. The processing unit may further be connected to external imaging means, preferably an ultrasound probe for controlling the system aided by detected image data of the external imaging means of the body duct and in particular a distal tip of the biomedical device.

The system may further have cardiac cycle detector for detecting a temporal change of a vascular pressure of the body duct (not shown in FIGS. 22A-22J). The cardiac cycle detector may be connected to the processing unit for controlling the actuation unit such that the puncturing element may be introduced into the body duct at a specific vascular pressure of the body duct.

The pressure sensor 51 which may be arranged in the hemostasis valve 52 may be adapted to detect if the puncturing element 41 was introduced into the body duct, in particular by being connected to the processing unit. The processing unit may be adapted to control the actuation unit to prevent further advancement of the puncturing element 41 once it was the pressure sensor 51 detects a pressure change corresponding to a successful puncturing of the body duct.

1.-41. (canceled)

42. A system for carrying out a percutaneous catheterization of a body duct through an insertion site of a patient's skin using a biomedical device having an introducer assembly, wherein the introducer assembly has:
a dilator for widening of an insertion site of a patient to access the body duct,
a sheath for introduction into the body duct, and
an inner guiding unit for guiding at least one of the dilator and the sheath into the body duct;
the dilator having a tapered outer section and at least one distal opening, and being arranged at least partially concentrically around the guiding unit and the sheath having at least one distal opening and being arranged or arrangeable at least partially concentrically around the dilator, such that the guiding unit, the dilator, and the sheath are movable relative to each other in a longitudinal direction of the introducer assembly;
wherein the system comprises at least one actuation unit which is configured to actuate at least one of the dilator, the sheath, the guiding unit, a puncturing element, an insertion guide and a guidewire of the guiding unit, of the device in a longitudinal direction of the introducer assembly; and
wherein the system comprises a shearing unit which is actuatable to perform a sheared aperture at the insertion site.

43. The system according to claim 42, wherein the system has at least one mounting section, the mounting section being adapted for releasably mounting at least one part of the introducer assembly selected from at least one of: the dilator, the sheath, the guiding unit, a puncturing element, a guidewire, and an insertion guide, wherein the mounting section comprises an at least partially open shape in a radial direction of the system corresponding to the mountable part of the introducer assembly.

44. The system according to claim 43, wherein the system has an engagement member which is convertible between an engagement configuration and a disengagement configuration;
wherein the at least one mountable part of the introducer assembly in the engagement configuration is fixedly held within the mounting section, at least with respect to movement along the longitudinal direction relative to the mounting section; and
in the disengagement configuration, the at least one mountable part is detached from the mounting section, such that the mountable part can be removed from the system.

45. The system according to claim 42, wherein the actuation unit comprises a clutch comprising at least two rollers which have an adjustable interdistance, such that the actuation of the actuation unit can be switched between configurations for actuating at least two of: the dilator, the sheath and the guiding unit, the puncturing element, the guidewire, and an insertion guide.

46. The system according to claim 42, wherein the system comprises at least one guiding element such that at least one of the actuation unit and the at least one mounting section are movable along the guiding element.

47. The system according to claim 42, wherein the system comprises a processing unit which is adapted for control of at least one of the shearing unit, the actuation unit, and the engagement member.

48. The system according to claim 42, wherein the system comprises the biomedical device.

49. A system for carrying out a percutaneous catheterization of a body duct comprising a shearing unit, wherein the shearing unit comprises
at least one shearing element for performing a sheared aperture at an insertion site, and
an actuation unit which is configured for translationally moving the at least one shearing element parallel to a longitudinal axis of the shearing unit;
wherein the actuation unit is configured for moving the shearing unit to perform the sheared aperture by at least one of
(i) rotating the at least one shearing element around a longitudinal axis of the shearing unit moving the shearing elements radially outwardly with respect to a longitudinal axis of the shearing unit, and
(ii) moving the shearing elements radially outwardly with respect to a longitudinal axis of the shearing unit.

50. The system according to claim 49, wherein the system comprises a processing unit which is configured for operating the actuation unit and the shearing unit by repeated sequential translational movement of the shearing unit in a distal direction of the shearing unit and subsequent rotation of the shearing unit around the longitudinal axis;
wherein the at least one shearing element has a non-rotationally symmetric cross-sectional shape at its distal tip or is arranged radially offset to a rotational axis of the shearing unit and the at least one shearing element is adapted for performing a sheared aperture by blunt dissection.

51. The system according to claim 49, wherein the system comprises a blunt inserter which has a blunt distal tip and is configured to be insertable into an inner lumen of the shearing unit.

52. The system according to claim 49, wherein the system comprises an expansion element which is arrangeable between the first and a second shearing elements, wherein the expansion element is configured for moving the shearing elements radially outwardly with respect to the longitudinal axis of the shearing unit.

53. The system according to claim 49, wherein the shearing elements are pivotally connected at at least one fulcrum point for relative pivotal movement thereabout, and a conversion mechanism of the shearing unit is configured for converting a translational movement of an actuation section of the conversion mechanism into an outwardly pivoting movement of the shearing elements around the at least one fulcrum point for performing the sheared aperture.

54. The system according to claim 53, wherein the actuation unit is configured to actuate the conversion mechanism to convert the shearing elements between a closed configuration in which the shearing elements are adjacent each other and an open configuration in which the shearing elements are pivoted outwardly.

55. The system according to claim 54, wherein the system comprises a processing unit which is adapted for operating the actuation unit and the shearing unit by repeated sequential movement of the shearing unit in the distal direction of the shearing unit in the closed configuration and subsequent conversion of the shearing unit to the open configuration for performing a plurality of sheared apertures at an insertion site.

56. The system according to claim 49, wherein the shearing elements have distal ends which converge to form at least one distal tip for insertion into the patient's tissue at an insertion site when the shearing elements are in a closed configuration.

57. The system according to claim 49, wherein the shearing elements are configured such that respective blunt edges or cutting edges of the shearing elements face radially outwardly in a closed configuration.

58. A system comprising a biomedical device for percutaneous catheterization of a body duct wherein the device has an introducer assembly having
a dilator for widening of an insertion site of a patient to access the body duct,
a sheath for introduction into the body duct, and
an inner guiding unit for guiding at least one of the dilator and the sheath;
the dilator having a tapered outer section, a distal opening, and being arranged at least partially concentrically around the guiding unit;
the sheath having a distal opening and being arranged at least partially concentrically around the dilator, such that the guiding unit, the dilator, and the sheath are movable relative to each other in a longitudinal direction of the introducer assembly; and
wherein the system comprises at least one actuation unit which is configured to actuate at least one, two, or all of the dilator, the sheath, the guiding unit, a puncturing element, a guidewire, and an insertion guide, of the device in a longitudinal direction of the introducer assembly.

59. The system according to claim 58, wherein the system comprises one actuation unit which comprises a clutch comprising at least two rollers which have an adjustable interdistance, such that the actuation of the actuation unit can be switched between configurations for actuating at least two, three, or all of: the dilator, the sheath and the guiding unit, the puncturing element, the guidewire, and an insertion guide.

60. A system comprising a biomedical device for percutaneous catheterization of a body duct,
wherein the device has a positionable introducer assembly for puncturing an application site at a patient's skin and an external imaging means for imaging a body duct of a patient in two dimensions or three dimensions, optionally in a time periodic measurement;
wherein the external imaging means and the introducer assembly are connected to a common support of the system.

61. The system according to claim 60, wherein the system comprises an external guidance means, wherein the guidance means have at least one of
a stereo camera or a RGBD camera,
a LIDAR system, and
an electromagnetic sensor;
such that one of a distance of the patient to the imaging means or the introducer assembly and a surrounding of the patient to the imaging means or the introducer assembly can be determined or reconstructed.

62. The system according to claim 60, wherein the guidance means are one of:
immovably connected to the external imaging means,
immovably connected to the patient or a stationary surface, and
attached to a movable surface which is independently movable from the imaging means.

63. The system according to claim 60, wherein the device comprises an introducer positioner for positioning at least one of the introducer assembly relative to the body duct and the common support;
wherein the introducer positioner is configured to position the introducer assembly in one of:
two translational degrees of freedom and at least one rotational degree of freedom, and
three translational degrees of freedom and at least one rotational degree of freedom.

64. The system according to claim 60, wherein the common support is
a hand-held support for manual alignment of the device relative to the patient,
a spatially fixed support which comprises a prepositioning means, or
a spatially movable support which comprises a device positioner for positioning of the device relative to the body duct independently in two or three translational degrees of freedom and at least one of one, two, or three rotational degrees of freedom.

65. The system according to claim 60, wherein the system comprises an force sensor which is configured for converting an external force exerted on at least a subsection of the system (103) to an electrical signal indicative of the external force.

66. The system according to claim 60, wherein the system comprises a processing unit for processing data relating to at least one of:
the measured image of the body duct of the external imaging means,
the determined or reconstructed distance of the guidance means, and
the electrical signal of the force sensor;
to calculate, based on the data, at least one electrical signal for:
operating at least one of the introducer positioner and the device positioner.

67. The system according to claim 66, wherein the system comprises a memory unit connected or connectable to the processing unit for saving data relating to at least one of:
the measured image of the body duct of the external imaging means,
the determined or reconstructed distance of the guidance means, and
the electrical signal of the force sensor.

68. The system according to claim 52, wherein the expansion element is formed by an inflatable expansion element or an expansion element which is configured to be insertable into an inner lumen of the shearing unit.

69. The system according to claim 54, wherein the actuation unit is adapted to advance and retract the shearing unit towards and away from an insertion site.

70. The system according to claim 60, wherein the external imaging means are an ultrasound probe, a Doppler ultra-sound probe, or an X-ray imaging probe.
